(19) United States
(12) Patent Application Publication
(10) Pub. No.: US 2019/0008674 A1
(43) Pub. Date: Jan. 10, 2019

(54) DEVICES AND METHODS FOR PREVENTING PRETERM BIRTH

(71) Applicant: THE TRUSTEES OF COLUMBIA UNIVERSITY IN THE CITY OF NEW YORK, New York, NY (US)

(72) Inventors: Kristin M. MYERS, New York, NY (US); Michael J. FERNANDEZ, Houston, TX (US); Joy Y. VINK, New York, NY (US); Ronald J. WAPNER, Media, PA (US); Andrea R. WESTERVELT, New York, NY (US)

(21) Appl. No.: 16/118,987

(22) Filed: Aug. 31, 2018

Related U.S. Application Data
(63) Continuation-in-part of application No. PCT/US2017/020624, filed on Mar. 3, 2017.
(60) Provisional application No. 62/303,689, filed on Mar. 4, 2016.

(51) Int. Cl. A61F 6/08; A61B 17/42; A61B 5/00

(52) U.S. Cl. CPC A61F 6/08; A61B 17/42; A61K 9/0036; A61B 2017/4225; A61B 5/4331

(57) ABSTRACT

A pessary and associated methods for preventing a preterm birth includes a pessary body having a first end and a second end offset from one another. The first end defines a first opening that is configured to receive a cervix. The pessary body has an exterior wall that extends from the first end towards the second end. The exterior wall has an outer surface, and an inner surface opposite the outer surface, the inner surface enclosing a channel that extends between the first and second ends and that is in fluid communication with the first opening. The pessary body further has an interface surface that extends between the first end and the second end. The interface surface is configured to engage the cervix so as to secure the pessary to the cervix.

### CROSS REFERENCE TO RELATED APPLICATION

This application is a Continuation-In-Part application of International Patent Application No. PCT/US2017/020624, filed Mar. 3, 2017, which claims priority to and the benefit of U.S. Provisional Application No. 62/303,689, “Devices and Methods for Minimizing Likelihood of Preterm Birth” (filed on Mar. 4, 2016), the disclosures of both of which are hereby incorporated by reference in their entireties for any and all purposes.

### GOVERNMENT RIGHTS

This invention was made with government support under DGE-11-44155 awarded by the National Science Foundation. The government has certain rights in the invention.

### TECHNICAL FIELD

This application relates to methods for preventing spontaneous preterm birth (sPTB) and devices associated therewith.

### BACKGROUND

Preterm birth (PTB) is defined as delivery before 37 weeks of gestation and after 20 weeks. About 20% of these early births result from a medically indicated necessity, determined by the patient's physician, and can include maternal distress factors (e.g., preeclampsia or placenta previa) or fetal distress factors (e.g. oligohydramnios (deficiency of amniotic fluid) or growth restriction). The remaining 80% of PTBs result from spontaneous preterm birth (sPTB) and typically cannot be predicted unless a patient has a history of sPTB. sPTB can be broadly divided into three general categories: cervical insufficiency; premature preterm rupture of membranes; or preterm labor. A more current understanding is that sPTB is a complex continuum involving multiple phenotypes and diverse factors. PTB is a major long-lasting public health problem with heavy emotional and financial consequences for families and society. Statistics released in November 2014 by the World Health Organization report that PTB globally affects 15 million babies annually, is the leading cause of childhood (<5 years old) death, and in 2013 was responsible for 1 million deaths. For infants that survive, PTB is a leading cause of long-term disabilities. The lower the gestational age at birth the longer the hospital stay and the greater the cost and risk of long term sequelae.

Despite extensive research, strategies to address known risk factors of sPTB in early pregnancy (e.g., genitourinary infection and poor nutrition) have been ineffective, as have drug therapies targeted against uterine contractions, infection, or inflammation.

### SUMMARY

There is a need in the art for devices and related methods for reducing the incidence of PTB and sPTB. In some aspects of the present disclosure, a pessary and associated methods that prevent preterm birth includes a pessary body having a first end and a second end offset from one another. The first end defines a first opening configured to receive a cervix. The pessary body has an exterior wall that extends from the first end towards the second end. The exterior wall has an outer surface, and an inner surface opposite the outer surface, the inner surface enclosing a channel that extends between the first and second ends and that is in fluid communication with the first opening. The pessary body further has an interface surface that extends between the first end and the second end. The interface surface is configured to engage the cervix so as to secure the pessary to the cervix.

In other aspects of the present disclosure, a method for preventing preterm birth in a patient is described. The method includes measuring a first angle defined by an axis defined by a uterine longitudinal axis and a cervical axis. The uterine longitudinal axis is defined by a length of the patient's uterus and the cervical axis is defined by a line along with the patient's cervical opening extends. The method also includes measuring a cervical diameter of a patient. Additionally, the method includes measuring at least one height of a vaginal canal of the patient. Based on the steps of measuring the angle, measuring the cervical diameter, and measuring the height, a pessary may be designed.

In other aspects of the present disclosure, a method of predicting a likelihood of preterm birth in a patient is disclosed. The method includes generating a computer simulation of a mechanical environment of pregnancy from a series of maternal anatomical measurements. The maternal anatomical measurements are derived from a series of ultrasound-based images. The method also includes predicting a likelihood of preterm birth if one or more factors characteristic of increased risk of preterm birth are present in the computer simulation.

In still further aspects of the present disclosure, a method of predicting an amount of cervical stretch likely to occur in a subject during pregnancy is disclosed. The method includes performing ultrasound-based imaging of the subject to obtain a series of maternal anatomical measurements. The method further includes generating a computer simulation of a mechanical environment of pregnancy from the series of maternal anatomical measurements. The computer simulation represents a base-line for the patient. The method also includes applying an intrauterine pressure to the computer simulation of the mechanical environment and predicting the amount of cervical stretch that would result from the intrauterine pressure.

### BRIEF DESCRIPTION OF THE DRAWINGS

The summary, as well as the following detailed description, is further understood when read in conjunction with the appended drawings. For the purpose of illustrating the disclosed devices and methods, there are shown in the drawings exemplary embodiments of the devices and methods; however, the devices and methods are not limited to the specific embodiments disclosed. In the drawings:

FIG. 1 illustrates: A) a biomechanical model of pregnancy; B) an exemplary cervix at 90° to anterior lower uterine segment (LUS); C) an exemplary cervix at 100° to anterior LUS; and D) an exemplary cervix at 110° to anterior LUS.

FIG. 2 illustrates gestation time points of data collection for serial ultrasound and cervical aspiration and corresponding engineering assumptions of tissue remodeling;

FIG. 3 illustrates exemplary transabdominal ultrasound scans at 25 weeks gestation;

FIG. 4 illustrates exemplary transperineal ultrasound scans at 25 weeks of gestation;

FIG. 5 illustrates a parameterized CAD model of pregnancy in (A) and an exemplary FEA simulation of a nulliparous pregnant patient at 25 weeks based on ultrasonic measurements in FIGS. 3 and 4;

FIG. 6 shows OCT collagen directionality and dispersion of axial slices of (A) a nonpregnant human cervix and (B) a pregnant human cervix;

FIG. 7 shows fiber-based material model fits for human (A) a cervix and (B) an amnion layer of the fetal membrane, were λ1 is the elongation and λ2 is the lateral contraction;

FIG. 8 shows a top perspective view of a pessary according to one embodiment;

FIG. 9 shows a bottom perspective view of the pessary of FIG. 8;

FIG. 10 shows a cross-sectional side view of the pessary of FIG. 8;

FIG. 11 shows a schematic view of a patient's cervix without a pessary.

FIG. 12 shows a schematic cross-sectional view of the patient's cervix of FIG. 11 fitted with the pessary of FIG. 8;

FIG. 13 shows a schematic cross-sectional view of the patient's cervix of FIG. 11 fitted with a pessary according to another embodiment;

FIG. 14 shows a cross-sectional side view of the pessary of FIG. 13;

FIG. 15 shows a side view of a pessary according to another embodiment; and

FIG. 16 shows bottom views four pessaries according to various embodiments.

### DETAILED DESCRIPTION OF ILLUSTRATIVE EMBODIMENTS

The disclosed devices and methods may be understood more readily by reference to the following detailed description taken in connection with the accompanying figures, which form a part of this disclosure. It is to be understood that the disclosed devices and methods are not limited to the specific devices and methods described and/or shown herein, and that the terminology used herein is for the purpose of describing particular embodiments by way of example only and is not intended to be limiting of the claimed devices and methods.

Unless specifically stated otherwise, any description as to a possible mechanism or mode of action or reason for improvement is meant to be illustrative only, and the disclosed devices and methods are not to be constrained by the correctness or incorrectness of any such suggested mechanism or mode of action or reason for improvement.

Throughout this text, the descriptions refer to devices and methods of using said devices. Where the disclosure describes or claims a feature or embodiment associated with a device, such a feature or embodiment is equally applicable to the methods of using said device. Likewise, where the disclosure describes or claims a feature or embodiment associated with a method of using a device, such a feature or embodiment is equally applicable to the device.

When a range of values is expressed, another embodiment includes from the one particular value and/or to the other particular value. Further, reference to values stated in ranges includes each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

Certain features of the disclosed devices and methods which are, for clarity, described herein in the context of separate embodiments, may also be provided in combination in a single embodiment. Conversely, various features of the disclosed devices and methods that are, for brevity, described in the context of a single embodiment, may also be provided separately or in any subcombination.

As used herein, the singular forms “a,” “an,” and “the” include the plural.

The term “comprising” is intended to include examples encompassed by the terms “consisting essentially of” and “consisting of”; similarly, the term “consisting essentially of” is intended to include examples encompassed by the term “consisting of.”

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “administering to said patient” and similar terms indicate a procedure by which the disclosed pessaries are placed at the opening of the cervix.

The term “subject” as used herein is intended to mean any animal, in particular, mammals. Thus, the methods are applicable to human and nonhuman animals, although preferably used with mice and humans, and most preferably with humans. “Subject” and “patient” are used interchangeably herein.

The following abbreviations are used throughout the specification and claims: anterior cervical angle (AUCA); posterior cervical offset (PCO); preterm birth (PTB); lower uterine segment (LUS); spontaneous preterm birth (sPTB).

Mechanical functions of the uterus, fetal membrane, and cervix have dynamic biological and mechanical roles to protect the fetus during full gestation and to allow the safe passage at time of delivery. For example, appropriate mechanical function of the cervix is required to maintain the developing fetus. The mechanical deformation of the cervix is believed to be the final common pathway for many etiologies of sPTB. sPTB related to cervical dysfunction is often called cervical insufficiency, which is the painless gradual dilation of the cervix resulting in a sPTB, and it is hypothesized to be the result of premature cervical remodeling. This mechanical role of the cervix is recognized clinically. Both risk assessment and management of sPTB rely heavily on a surrogate measure of cervical mechanical function, ultrasonic cervical length. The serial ultrasound assessment of cervical length is an integral component in evaluating a woman's risk for sPTB.

Clinically, a short cervix, which is associated with an increased risk of sPTB, is a cervix less than 25 mm in length. Taken alone, the predictive capability of a sonographic short cervix is unclear, with some studies citing early assessment between 16 to 18 weeks with serial evaluations is the best predictor of sPTB.

Risk factors for preterm birth may be assessed by ultrasound. To assess these risk factors, ultrasound can be performed using one or more of a transabdominal probe, a transperineal probe, a transvaginal probe, or a combination thereof. Specifically, ultrasound can be used to obtain a series of maternal anatomical measurements which can be used to evaluate risk factors for preterm birth. Exemplary maternal anatomical measures include, for example, placenta location, placenta volume, fetal biometrics, amniotic fluid index, uterine diameter, uterine thickness, cervical length, cervical diameter, cervical angle with anterior lower uterine segment, cervical os, mechanical load, tissue stretch, or a combination thereof. Uterine diameter measurements may include, for example, a longitudinal uterine diameter, an anterior-posterior diameter, a transverse uterine diameter, or a combination thereof.

Effectiveness of current strategies to minimize the likelihood of sPTB, including progesterone treatment and strategies aimed at restoring the mechanical function of the cervix (cervical cerclage, a surgical suture, pessaries, etc.), are unclear due to difficulties associated with identifying high-risk women. Disclosed herein are methods that enable identification of women who are at the highest risk for sPTB. Conversely, these methods may be used to rule out women at the lowest risk of sPTB to avoid unnecessary and costly interventions. These methods identify women early in pregnancy to develop etiology-specific and patient-specific interventions.

The disclosed methods can be used to create a predictive finite element (FE) model of the mechanical environment of pregnancy based on the fewest and most minimally-invasive clinical measurements possible. The disclosed methods can be used, for example, to minimize the likelihood of preterm birth by predicting a likelihood of preterm birth, predicting an amount of cervical stretch likely to occur in a subject during pregnancy, and determining the amount of cervical angle adjustment needed to minimize the likelihood of preterm birth.

The disclosed methods also include using a pessary to adjust a cervical angle and minimize cervical shortening in a pregnant patient. In some embodiments, the methods comprise: performing ultrasound-based imaging of the patient to obtain a series of maternal anatomical measurements; generating a computer simulation of a mechanical environment of pregnancy from the series of maternal anatomical measurements; and, based on the series of maternal anatomical measurements, administering, selecting, or fabricating a pessary to adjust a cervical angle and minimizing cervical shortening in a pregnant patient.

Maternal anatomical measurements derived from a series of ultrasound-based images may be used to predict a likelihood of preterm birth if one or more factors characteristic of increased risk of preterm birth are present in the computer simulation. Maternal anatomical measurements may also be used to predict an amount of cervical stretch likely to occur in a subject during pregnancy. In some embodiments, the methods can comprise: performing ultrasound-based imaging of the subject to obtain a series of maternal anatomical measurements; generating a computer simulation of a mechanical environment of pregnancy from the series of maternal anatomical measurements, the computer simulation representing a base-line for the patient; and predicting the amount of cervical stretch that would result from intrauterine pressure. For example, the intrauterine pressure used to predict cervical stretch may be a theoretical intrauterine pressure, or it may be measured in the patient.

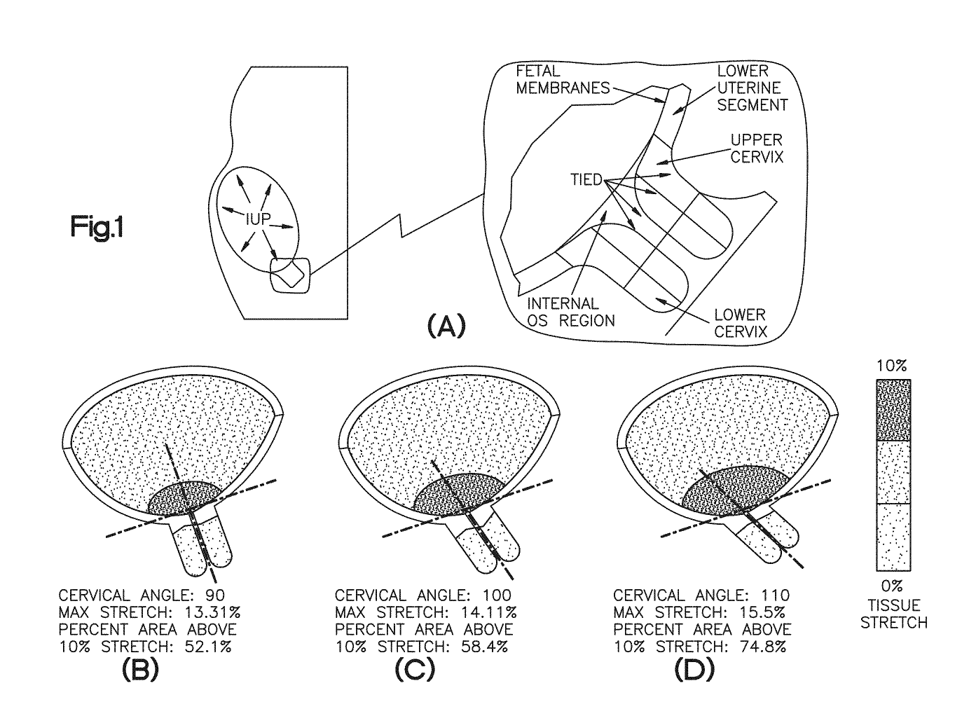

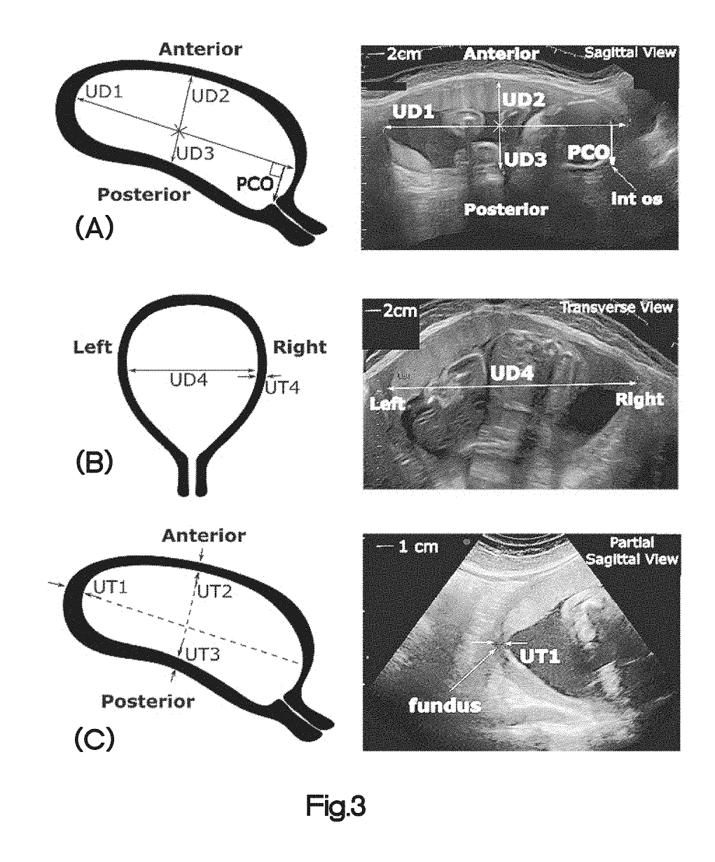

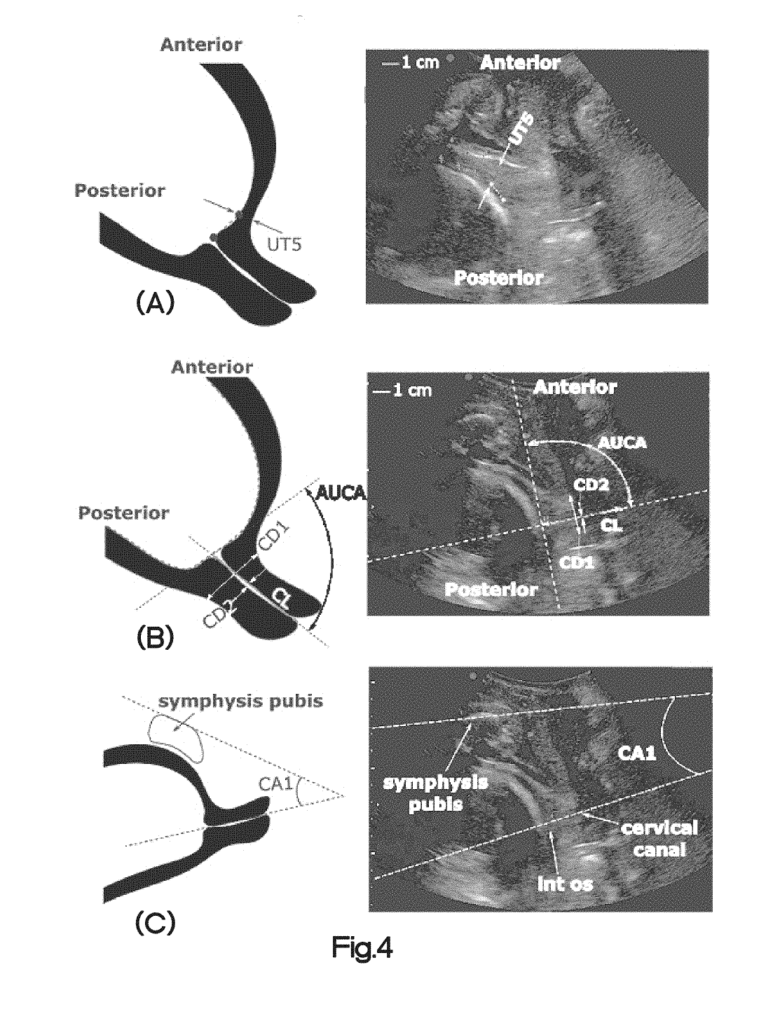

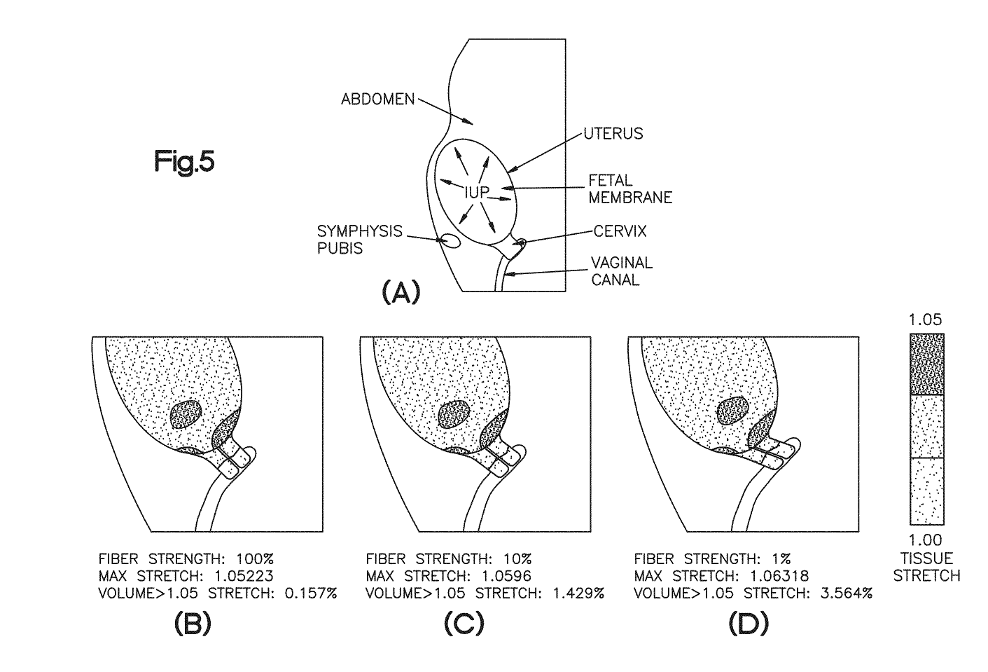

Computer simulations can be generated, for example, using a parametric scripting code (such as Python™) written to generate an analytical geometric representation of the pregnant abdomen. Ultrasonic measurements (FIG. 3 and FIG. 4) may be used as input parameters to generate an analytical geometric representation of the pregnant abdomen (using for example, Trelis Pro 15.1, SolidWorks, or other modeling software). Geometry of the uterus, cervix, fetal membrane, vaginal canal, and abdomen may be generated by using Boolean addition and subtraction operations on geometric primitives (FIG. 5, (A)).
The uterus can be formed by transforming two spherical surfaces to form ellipsoids presenting the outside and inside uterine walls. The interior uterus can be scaled to the diameters obtained during ultrasound and rotated in relation to the reference angle of the symphysis pubis. In some embodiments, an arbitrary value of 15° can be used. The outer shell can be scaled, translated, and/or rotated to accommodate differences in uterine wall thickness in the anterior-posterior, superior-inferior, or left-right directions.
The cervix can be built using a hollow cylinder according to clinical cervical dimensions. For example, the cervix can be built by creating a cylinder representing the diameter of the inner canal and subtracting that volume from a larger cylinder representing the outer cervical diameter and cervical length. The resultant hollow cylinder can then be moved and rotated according to posterior cervical offset (PCO) and anterior cervical angle (AUCA). The cylinder can be rounded at its corners to match the anatomical rounding of the uterocervical junction, and to replicate the roundness of the most exterior end of the cervix (i.e. external os). Its edges can be blended at both ends to eliminate non-anatomical corners and to match the clinical presentation of the uterus-cervix connection.
For purpose of tissue loading analysis, the cervix can then be separated into three different regions: an upper portion, a lower portion, and the internal os region (FIG. 1, (A)). First, the cylindrical representation of the cervix can be cut by a plane normal to the external os at a fixed distance of 15 mm from the internal os. Second, the top portion of the cervix can then be separated by a surface extended from a smaller cylinder with a diameter that was twice of the cervical inner canal.
The curved shape of the vaginal canal can be formed by fitting a spline to three points on the outer cervical os and one point at the approximate location of the vaginal introitus.
The fetal membrane can be generated with uniform thickness in a similar manner to the uterus. The outside of the fetal membrane volume shares an ellipsoidal surface primitive with the inner uterine wall.
   Geometric features of the model can be coupled mathematically so that the modification of anatomical values will still result in congruent geometry. The uterus, cervix, and abdomen can be meshed with an adaptive tetrahedral meshing algorithm (including, but not limited to, Trelis Pro 14.1 or v15.1.3, csimsoft LLC), and the fetal membrane volume can be meshed manually as a single layer of hexahedral elements. In an alternative to Trelis, the pregnancy model can be generated as a three-dimensional geometry using SolidWorks modeling software, the three-dimension geometry can be exported into Materialise 3-Matic software to create a finite element mesh, and the mesh geometries can be imported into FEBio software for finite element analysis. It will be understood that other modeling and/or finite element analysis software can be used.

Specific hyperelastic material constitutive models for the uterus, cervix, and fetal membrane may be derived that account for tissue ultrastructure and cervical growth and remodeling (G&R). Tissue material behavior may be a function of tissue ECM and muscle fiber ultrastructure using quasi-static forms of the constitutive equation for the cervix and the fetal membrane. For this initial modeling framework, short-term time-dependent material characteristics may not be used, such as visco- or poro-elastic contributions that occur on time-scales much smaller than the gestational weeks of pregnancy. Active uterine contractions may also not be considered.

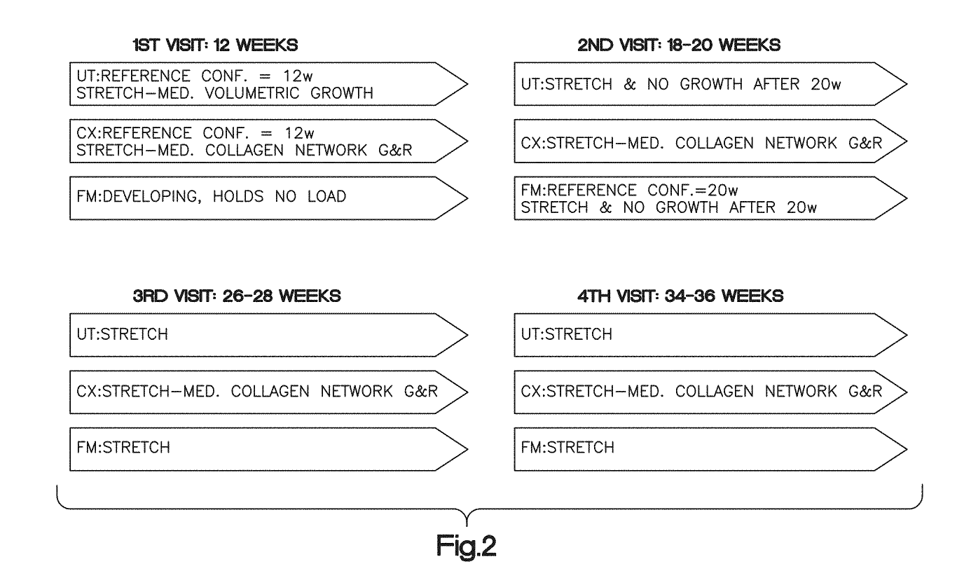

To capture the temporal changes in the material behavior of both the uterus and cervix a stretch-mediated approach is used to derive, implement, and quantify tissue growth and remodeling (G&R) characteristics using. With reference to FIG. 2, using simplified engineering assumptions, the postulated constitutive equations detailed below offer a starting framework. As experimental evidence is collected the forms of these equations may either be validated or alternative modeling equations may be employed. In FIG. 2, the uterus, cervix, and fetal membrane are referred to with the abbreviations UT, CX, and FM, respectively.

The uterus may be treated as a passive fibrous muscular material between 12 and 36 weeks of pregnancy when major active uterine contractions are typically suppressed. At week 12, the material is in its stress-free reference configuration. From week 12 to week 20, the volumetric growth of the material may be described kinematically through a multiplicative decomposition of the deformation gradient the material kinematically (F=F^{e}F^{g}) into an irreversible inelastic growth component F^{g} and a reversible elastic component F^{e}. In this approach, the traction-free reference configuration may be recast into a heterogeneous body using a kinematic description for growth F^{g} that requires an elastic deformation F^{e} to ensure material compatibility. One may evolve the growth tensor F^{g} by \dot{F}^{g}=D^{g}F^{g}, where D^{g} is the growth rate tensor (i.e. the symmetric part of the velocity gradient of F^{g}). As an initial assumption, one may constitutively prescribe an isotropic growth rate Dᵢₛₒ^{g}=\dot{α}I where \dot{α} is a linear scalar function of an effective elastic stretch measure λ_{eff}=\sqrt{tr(B^{e})/3}. This linear scalar function may be calibrated to match the volumetric growth as viewed in ultrasound data. Beyond 20 weeks the uterus will stretch elastically.

The uterine elastic material response may be modeled using a decoupled form of the strain energy density presented by Blemker et al. and with specification to the uterine muscle fiber ultrastructure. The de-coupled form of the strain energy accounts for tissue muscle fiber architecture, individual fiber properties, and shear properties along and transverse to the fiber. The material strain energy density is a function of the modified deviatoric stretch invariants Ī₄ and Ī₅, the square of the fiber stretch and the fiber interaction term, respectively. They are recast into the strain invariants

$${B}_{1} = \sqrt{\frac{{\overline{I}}_{5}}{{\overline{I}}_{4}} - 1} and {B}_{2} = {cosh}^{- 1} ( \frac{{\overline{I}}_{1} {\overline{I}}_{4} - {\overline{I}}_{5}}{2 \sqrt{{\overline{I}}_{4}}} ) ,$$

to capture the shear components along and transverse to the fiber. Lastly, the fiber strain energy density may be phenomenologically prescribed as a piecewise exponential, and the directionality of the 3 discrete fiber families may be specified based on optical coherence tomography (OCT) fibers maps of ex vivo tissue.

To obtain baseline measures of uterine material properties, specifically to capture muscle fiber architecture, and to understand the range in material characteristics between the nonpregnant and pregnant tissue, ex vivo uterine tissue samples may be mechanically tested and OCT imaged. Samples may be taken from nonpregnant patients who are undergoing total hysterectomy for benign indications and pregnant patients who are undergoing a cesarean hysterectomy at term. Whole cervix and uterus samples may be collected from premenopausal, nonpregnant (n=20) women without a history of preterm birth. Whole cervix and uterus tissue pregnant samples (n=10) may also be collected from patients undergoing a caesarean hysterectomy due to postpartum hemorrhage/atony or abnormal (placenta previa) or invasive placentation (only cases where the accreta does not involve the cervix). Additionally, to examine normal pregnant uterine tissue lower uterine segment tissue samples (approx. 6×12 mm, approx. 4 mm thick, n=10) may also be collected from normal full-term cesarean section patients. While some of these tissues samples are not considered normal tissue, tissue that is sufficiently far from the site of known tissue pathologies may be analyzed.

Ex vivo tissue samples may first be analyzed for muscle fiber architecture via OCT by adapting a scanning protocol previously used to track myofibers in cardiac tissue. Once fiber directionality is mapped, a series of multi-axial mechanical tests using a universal material tester (Instron 5948 Microtester) coupled with a 3D digital image correlation system (DIC) (Correlated Solutions Inc., Columbia, S.C.) may be conducted. First, a series of spherical indentation measurements on whole tissue pieces may be performed, recording force-displacement and the 2D strain field of the tissue deformation against a flat rigid substrate. The tissue may then be cut into tensile strips parallel and off-angle to known fiber directions. The tissue may be pulled in a series of load-unload and ramp-hold tensile experiments, recording axial force-displacement and 3D strain fields of the deformation. Inverse finite element analysis may be used to fit the material parameters of the muscle model to the experimental force-displacement and 2D and 3D strain data. The material model may be validated by predicting experimental material behavior not used to inform the model fitting.

Cervical tissue may be modeled as a hydrated fiber composite porous material where the interstitial pore space allows for the growth and removal of solid mass. A constrained mixture model approach may be used that considers multiple stress-free configurations of newly formed crosslinked collagen fibers. Hence the total stress in the solid components will be the sum of individual cervical ECM components that will have separate constitutive behaviors, separate rates of production and removal, and separate evolving natural configurations. The temporal change in cervical material behavior may be determined using the idea of stretch-mediated adaptive elasticity to formulate mass rate equations for the production and removal of collagen crosslinks. The mass supply rate may be cast in terms of its apparent mass density e^{xlink}, defined by the elemental solid mass divided by its elemental volume including the pore spaces. During the growth process, solid can be added or removed within the volume such that the overall material becomes less or more porous, respectively. As a consequence of this interstitial growth, the referential apparent mass density ρᵣ^{xlink}, will no longer be invariant and will be considered an ECM compositional state variable when constitutively prescribing equations for the free energy density. In the case of cervical remodeling, the collagen fiber stiffness becomes a function of ρᵣ^{xlink}, similar to early experimental results of bone showing that the Young's modulus is proportional to various powers of its apparent density and studies on mouse cervical tissue showing a shift in mature collagen crosslinking density in pregnancy.

New cervical crosslinked collagen networks are created and destroyed at different times throughout pregnancy. Hence, the natural (i.e. reference) configuration of these network generations as they form and degrade may be tracked. Assuming new collagen networks are created in a stress-free configuration, the mass supply rate \hat{ρ}^{xlink} to the solid from the interstitial space enters the mass balance equation as,

$$\frac{D {ρ}^{x link}}{{D}_{t}} + {ρ}^{x link} {divv}^{s} = {\hat{ρ}}^{x link} ,$$

where ρ^{xlink} is the current apparent density and v^{s} is the velocity of the solid. The mass rate equation may also be written as

$$\frac{D {ρ}_{r}^{x link}}{{D}_{t}} = {\hat{ρ}}_{r}^{x link} ( {λ}_{eff}^{k} - {λ}^{★} )$$

where D(⋅)/Di is the material time derivative in the spatial frame, ρᵣ^{xlink}=jₖρ^{k}, and \hat{ρ}ᵣ^{xlink}=j^{k}\hat{ρ}^{k}. ρᵣ^{xlink} is the mass supply rate based on its reference configuration and it is a function of the different between λ* and λ_{eff}^{k}=\sqrt{tr(B^{k})/3}, λ* is a stretch threshold that one may determine from literature evidence of cervical fibroblast stretch-sensitivity and λ_{eff}^{k}=\sqrt{tr(B^{k})/3} is an effective stretch measure in the cervical tissue which will be evaluated for each generation k.

The elastic response of cervix will be the sum of the interstitial fluid pressure, calculated based on the Donnon osmotic pressure provided by the fixed charge density of the enmeshed glycosaminoglycan content, and the elastic response of the solid material components. The solid material is composed of a continuously distributed anisotropic collagen fiber network and an isotropic neo-Hookean ground substance. Here, it may be assumed that the elastin-dominated neo-Hookean ground substance does not remodel throughout pregnancy and the collagen fibers remodel via formation and degradation of its mature crosslink density. Therefore, the deformation of the neo-Hookean material will track with the original reference configuration and the collagen network will contain multi-generations of new and old fibers. For each collagen network generation, the total stress in the collagen network may be derived from summing the individual fiber stresses, and the shape of the fiber distribution may be informed by ex vivo collagen ultrastructure OCT studies. The stress-strain behavior of an individual fiber may be phenomenologically prescribed via a standard power-law relationship that holds only tension, as described in, with a corresponding fiber stiffness that is a function of the apparent mass density of the collagen crosslinking density ρᵣ^{xlink}. This function may be determined based on mechanical and biochemical evaluation.

To obtain the range in material characteristics between the nonpregnant and term pregnant tissue and to develop structure-function relationships between collagen crosslinking density and collage fiber stiffness, multi-axial mechanical tests, biochemical assays, OCT imaging may be performed on whole cervical tissue samples taken from the hysterectomy samples as described above for the uterine tissue. A series of spherical indentation and multi-axial tensile tests, as described above for uterine tissue, on cervical samples taken from multiple sites and conducted in multiple anatomical directions may be conducted. The collagen fiber directionality and dispersion in multiple anatomical directions may be evaluated via OCT and collagen crosslinking density via liquid chromatography tandem mass spectrometry (LC-MSMS). Inverse analysis may be conducted to find the best-fit material parameters for the tissue samples and correlate these best-fit parameters to collagen crosslinking density. Aspiration mechanical tests may be performed using the in vivo tool on these whole cervical tissue specimens, and inverse finite element analysis may be used determine the relationship between aspiration closure pressure P_{cl}, collagen fiber stiffness, and collagen crosslinking density

The fetal membrane may be modelled using the quasi-static form of the equations as presented in Mauri et al. Briefly, the membrane is treated as a fiber composite material, with two continuously distributed planar fibers that are slightly pitched from the plane of the membrane and embedded in an isotropic, compressible neo-Hookean material. Again, we phenomenologically prescribe the material behavior of a single fiber via a standard power-law relationship that holds only tension, and the total stress in the collagen network may be derived from summing the individual fiber stresses. Multiple sites of the membrane may be mechanically tested via bulge inflation and tension.

Also provided are predictive computer simulation models of a mechanical environment of pregnancy, comprising: a series of ultrasound images; and a computer readable medium for use by a computer in modeling data from the ultrasound images. The series of ultrasound images can comprise one or more ultrasound images of placenta location, placenta volume, fetal biometrics, amniotic fluid index, uterine diameter, uterine thickness, cervical length, cervical diameter, cervical angle with anterior lower uterine segment, cervical os, mechanical load, and tissue stretch, or a combination thereof.

Ultrasound images for use in the disclosed predictive computer simulation models can be obtained using, for example, a transabdominal probe, a transperineal probe, a transvaginal probe, or a combination thereof. In some embodiments, the ultrasound images can be obtained using a transabdominal probe. In some embodiments, the ultrasound images can be obtained using a transperineal probe. In some embodiments, the ultrasound images can be obtained using a transvaginal probe. In some embodiments, the ultrasound images can be obtained using any combination of the above probes.

As a fetus grows, its weight stretches the cervix. In women with premature cervical shortening, cervical stretching starts at the internal os, which may be seen on ultrasound as a cervical funnel. A patient-specific 3D finite element (FE) computer simulation models of the pregnant uterus and cervix was constructed to understand how anatomical and tissue mechanical factors influence cervical tissue stretch and shortening.

With reference to FIG. 1, (A)-(D), FE models (e.g., in FEBio 2.3.1) were generated using a custom computer script (e.g., in Trelis Pro 15.1.3) based on input parameters from transabdominal and transperineal ultrasound measurements (GE Voluson E8). For the baseline model, uterine diameter, uterine thickness, cervical length, cervical diameter, and cervical angle with anterior lower uterine segment (LUS) were measured for a 35 y/o normal patient at 21 weeks (FIG. 1, (A) and (B)). Cervical angle was ranged from 90 to 110 degrees from the anterior LUS (FIG. 1, (B)-(D)). The uterus and cervix were modeled as collagenous composite materials, and the fetal membrane as a nonlinear hyperelastic material. Intrauterine pressure was applied over the physiological range for 21 weeks (0 to 1 kPa), and the cervical stretch at the internal os was evaluated.

The cervical angle of 90° with respect to the anterior LUS has the lowest simulated stretch concentration at the cervix internal os. Maximum tissue stretch and percent surface area of cervix above a 10% stretch threshold both increase directly with cervical angle (FIG. 1, (B)-(D)).

Cervical angle contributes to internal os tissue stretch. As cervical angle deviates from 90° with respect to the anterior LUS, simulated cervical stretch increases. These results may explain how mechanical therapies such as a pessary change cervical angle and may contribute to minimizing the likelihood of preterm birth.

Geometric measurements of the uterus, cervix, and their position relative to bony reference landmarks may be measured via ultrasound (GE Voluson E8). All measurements may be taken with the transabdominal probe either in the transabdominal or transperineal position. At the earlier gestation time points, if cervical canal cannot be viewed, images may be taken with the transvaginal probe. All measurements may be taken with a void bladder with the patient lying down. A subset of low-risk patients (n=20) may also have ultrasound measurements repeated with a void bladder standing up.

The placenta location and volume, fetal biometrics, and amniotic fluid index may be measured and recorded. Next, overall uterine diameters (UDs) may be measured with the extended view imaging feature of the GE Voluson E8, which automatically registers adjacent ultrasound images as the probe is swept across the abdomen at a steady rate (errors in the this measurement may be assessed on a low-risk patient by assessing the measurement sensitivity to probe velocity and probe position relative to skin). To capture a sagittal view of the entire uterus, the transabdominal probe may be swept along the patient's abdomen from the fundus to the pubic bone at a rate of 2 cm/second. From this view the longitudinal uterine diameter (UD1), anterior-posterior diameter (UD2+UD3), and the cervical os offset from the longitudinal diameter (PCO) (FIG. 3, (A)) may be measured. To capture a transverse uterine diameter (UD4), the probe may be swept from left to right across the midabdomen and measure at its widest point (FIG. 3, (B)).

Uterine wall thicknesses (UT1-5) may then be measured at multiple locations from the fundus to the lower uterine segment (LUS) with the transabdominal probe in a standard clinical resolution (FIG. 3, (C)). The wall thickness (UT1-UT5) may be considered the echogenic signal from the serosa to the decidua (FIG. 3, (C) and FIG. 4, (A)). Attention may be given to the lower uterine wall thickness where UT5 may be measured at 1 cm increments from the cervical internal os using the perineal scan (FIG. 4, (A)). Cervical length (CL), cervical diameter (CD1), canal width (CD2), angle with the anterior lower uterine segment (AUCA), and angle with periosteum of the symphysis pubis (CA1) may also be assessed via the transperineal scan (FIG. 4, (B) and-(C)).

After the ultrasound, a speculum may be placed and 3 vaginal fluid samples may be collected to rule out vaginal infections, to measure fetal fibronectin levels, and to measure vaginal fluid pH. Samples may be analyzed via standard clinical assays.

The mechanical environment of pregnancy may be modeled after the drastic initial hormone-level increase that occurs within the first 12 weeks of pregnancy and before the drastic functional decline of the steroid hormone progesterone that happens within days of parturition to activate uterine contractility.

The following engineering assumptions may be made during modeling:
The uterus volumetrically grows from 12 to 20 weeks and stretches with no growth after 20 weeks; and
The fetal membrane stretches with no growth after 20 weeks;

Meshed anatomy from the experimental data taken at 12 weeks may be imported into FEBio, an NIH-funded open-sourced finite element code (url: http://febio.org) (as described in G. Ateshian, et al., Modeling the Matrix of Articular Cartilage Using a Continuous Fiber Angular Distribution Predicts Many Observed Phenomena. J Biomech Eng, 131(6):061003, 2009 (PMID: 19449957) and S. Maas, et al., FEBio: Finite Elements for Biomechanics. J Biomech Eng, 134(1):011005, 2012 (PMID: 22482660)). Appropriate boundary conditions and contact definitions may be applied, including gravity, the friction of the amniotic sac against the uterine wall, contact between uterine wall and abdomen, and contact between the cervix and the vaginal wall. FE formulations may be implemented for the proposed material equations using FEBio plug-in C++ object-oriented scripting platform. With the 12 week geometry, fiber directionality may be assigned to the uterus, cervical, and fetal membrane based on the OCT bench tests and previous work. For the uterus, material parameters may be assigned based on the average of nonpregnant values measured from the ex vivo tissue samples, and for the fetal membrane patient-specific measurements obtained from the delivered membrane may be used. For the cervix, the fiber-based material parameters corresponding to the aspiration closure pressure pcl established from the relationships derived from the ex vivo tissue studies may be assigned. As a simplifying assumption, the entire cervix may be homogeneously remodeled according to the stretch-levels achieved at the internal os. Loading conditions may be applied based on the patient-specific fetal biometry, amniotic fluid index, and a database of previously measure IUPs (N. M. Fisk, et al., Normal amniotic pressure throughout gestation. BJOG, 99(1):18-22, January 1992 (PMID: 1547165)) and use an inverse optimization method to calibrate the material growth rates for the uterus (α) and cervix (\hat{ρ}^{xlink}) such that the FE predictions of tissue deformation and cervix remodeling match the experimental time-course ultrasonic deformation and aspiration measurements.

The FE implementation may be validated by two mechanisms. First, for a subset of patients, maternal anatomy measurements at a single time point with the patient lying down and standing up may be used. Therefore, an attempt to predict the geometry of the patient standing up using the best-fit material parameters determined from the lying down position may be made. To validate the growth rate laws, a prediction of the tissue deformation and cervical remodeling at the later time points in pregnancy that are not used to inform the material models may be attempted.

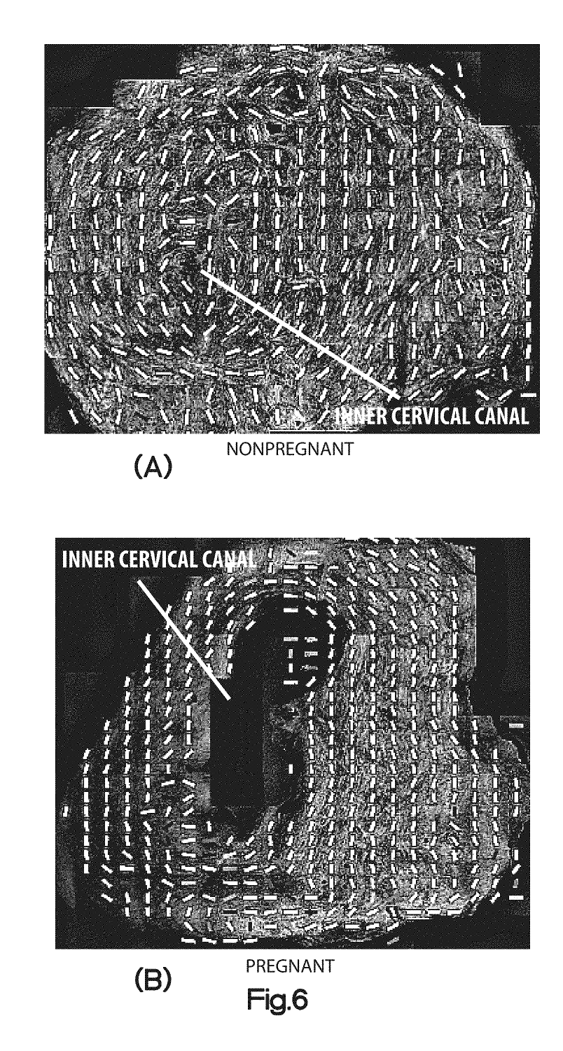

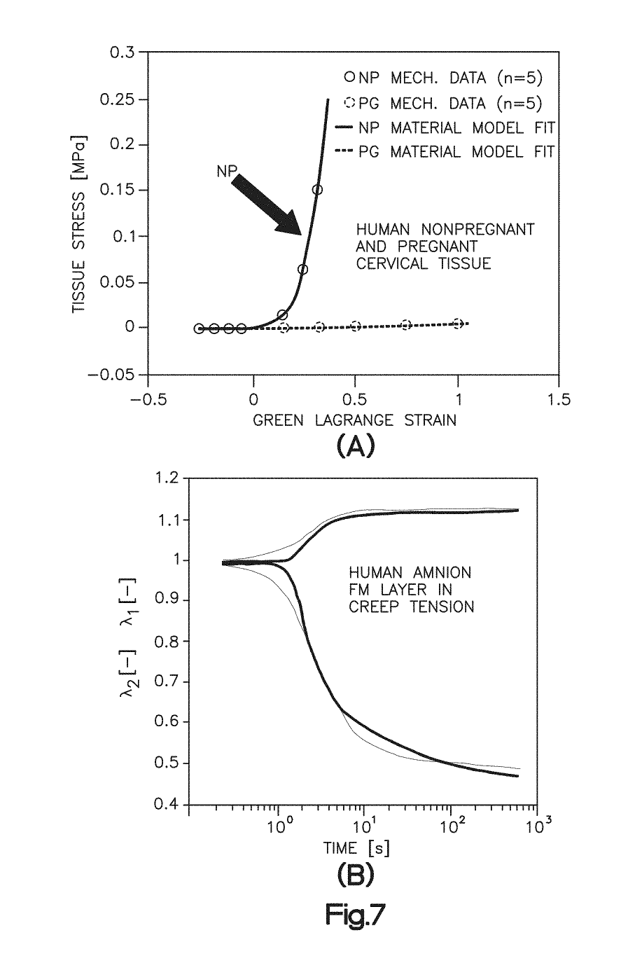

An ultrasound-based parametric CAD model of the pregnant abdomen was developed (FIG. 5, (A)) using the list of ultrasonic parameters outlined in FIG. 3 and FIG. 4. FE simulations were demonstrated using cervical and uterine fiber composite material parameters as disclosed in M. Fernandez, et al., Investigating the mechanical function of the cervix during pregnancy using finite element models derived from high-resolution 3D MRI. Comput Methods Biomech Biomed Engin, pages 1-14, May 2015 (PMID: 25970655) and K. M. Myers, et al., A continuous fiber distribution material model for human cervical tissue. J Biomech, 48(9):1533-1540, June 2015. (PMID: 25817474) and treating the fetal membrane as a nonlinear Ogden material fit to previously published mechanical data ad described in W. Buerzle et al., On the deformation behavior of human amnion. J Biomech, 46(11):1777-1783, July 2013 (PMID: 2377781). The outer abdomen was treated as a soft nearly incompressible neo-Hookean material with a modulus of 5 kPa. The maximum cervical stretch under an IUP=0.814 kPa, calculated for 25 weeks using IUP data as described in N. M. Fisk, et al., Rodeck. Normal amniotic pressure throughout gestation. BJOG, 99(1):18-22, January 1992 (PMID: 1547165), for three different scenarios of cervical collagen fiber stiffness was compared. As this single material parameter was decreased, the magnitude of tissue stretch increased at the location of the internal os and the cervical tissue volume fraction above a theoretical remodeling activation threshold of 1.05 increases. A continuously-distributed fiber composite material model for human cervical tissue based on initial OCT collagen fiber maps of orientation and dispersion was derived (FIG. 6-7). The OCT signal indicated an increase in dispersion for the pregnant tissue (data not shown), indicating remodeling activity. However, the drastic softening of the pregnant tissue was accommodated by a significant decrease in the intrinsic stiffness of the collagen fibers (FIG. 7, (A)). The extreme in-plane and thickness contraction observed in multi-axial tensile experiments was reproduced using a quasi-isotropic fibers distribution in the plane of the membrane, with a small pitch angle out-of-plane. (FIG. 7, (B)).

Maternal anatomical measurements can comprise one or more of placenta location, placenta volume, fetal biometrics, amniotic fluid index, uterine diameter, uterine thickness, cervical length, cervical diameter, cervical angle with anterior lower uterine segment, cervical os, mechanical load, and tissue stretch. Ultrasound-based imaging for use in the disclosed methods can include imaging performed with a transabdominal probe, a transperineal probe, a transvaginal probe, or a combination thereof. In some embodiments, the ultrasound-based imaging can be performed with a transabdominal probe. In some embodiments, the ultrasound-based imaging can be performed with a transperineal probe. In some embodiments, the ultrasound-based imaging can be performed with a transvaginal probe. In some embodiments, the ultrasound-based imaging can be performed with any combination of the above probes.

Generating a computer simulation of a mechanical environment of pregnancy from the series of maternal anatomical measurements may be performed as described above according to combinations of the steps described. The methods described for determining maternal anatomical measurements may also be used to determine whether administration of a pessary would minimize a likelihood of preterm birth. Administration of a pessary may minimize the likelihood of preterm birth by adjusting the patient's cervical angle and minimizing cervical shortening as pregnancy progresses. In some instances, a pessary administered to the patient may be chosen or designed based on the patient's anatomical measurements as determined based on any combination of the methods described herein. Characteristics of exemplary pessaries are described below.

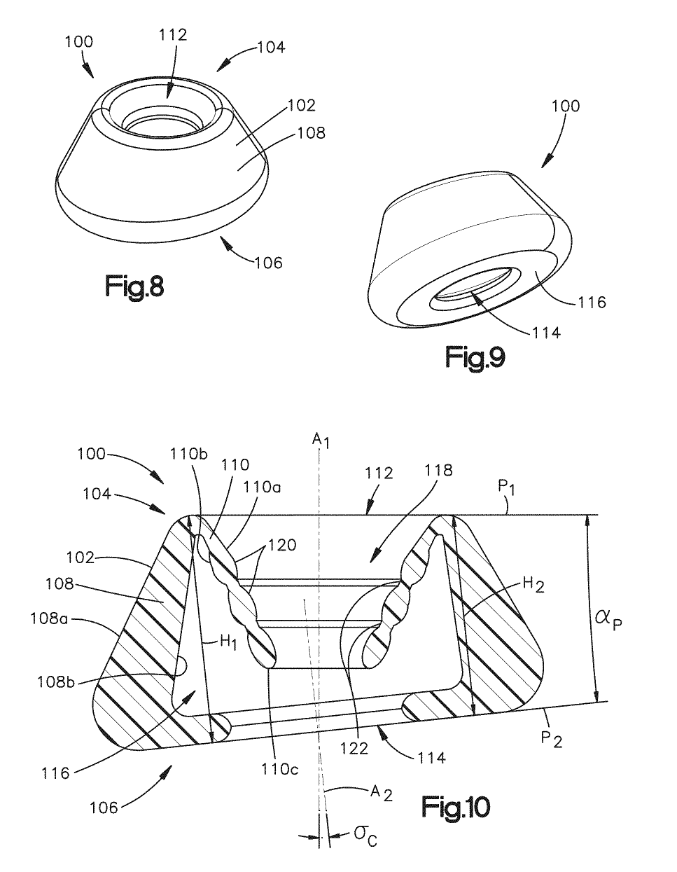

For example, FIGS. 8-12 show one embodiment of a pessary 100 that is configured to be supported around a patient's cervix to limit a likelihood of preterm birth. In general, the pessary 100 has a pessary body 102 that includes a first end 104 and a second end 106 that are offset from one another along a first direction. The first end 104 can be a superior end, and the second end 106 can be an inferior end. The pessary body 102 includes an exterior wall 108 that extends between the first end 104 and the second end 106. Further, the pessary body 102 includes an interior wall 110 that extends between the first end 104 and the second end 106. As will be described further below, the interior wall 110 is configured to engage a cervix 202 at the end of a uterine wall 204 of a patient so as to secure the pessary 100 to the cervix 202.

The first end 104 of the pessary body 102 defines a first or superior opening 112. The first end 104 can further define a first plane P₁. For example, the first end 104 can have an outermost surface that defines the first plane P₁. The first opening 112 can lie in the first plane P₁. The first opening 112 is sized and shaped to receive the patient's cervix 202 therein. In particular, the first opening 112 is configured to fit around a patient's cervix in order to hold the cervix 202 closed and limit cervical shortening as a pregnancy progresses. The first opening 112 can have any suitable shape. For example, in at least one embodiment, the first opening 112 can have a circular shape in the first plane P₁, and can have a central axis A₁. The central axis A₁ can be perpendicular to the first plane P₁. As another example, the first opening 112 can have an oblong shape.

The first end 104 includes a first perimeter in a first cross-sectional plane that is perpendicular to the central axis A₁. The first perimeter can have any suitable shape in the first cross-sectional plane. Further, the first end 104 can have a first area within the first perimeter. In some embodiments, the first perimeter can have a cross-sectional shape in the first cross-sectional plane that is circular. Further, the first end 104 and the first opening 112 can combine to define an annular shape. In alternative embodiments, the first end 104 can have another shape such as an oblong shape. Moreover, the cross-sectional shape of the first end 104 can be different from that of the first opening 112.

The second end 106 defines a second or inferior opening 114. The second end 106 can further define a second plane P₂. For example, the second end 106 can have an outermost surface that defines the second plane P₂. The second opening 114 can lie in the second plane P₂, and can have any suitable shape. For example, the second opening 114 can have an oblong or circular shape in the second plane P₂. The second opening can have a second central axis A₂. The second central axis A₂ can be perpendicular to the second plane P₂. The first and second planes P₁ and P₂ can be offset from one another by an acute, non-zero angle α_{P}. Thus, it can be said that the first and second planes P₁ and P₂ are angularly offset from one another and are non-parallel to one another. For example, the angle α_{P} between planes P₁ and P₂ can be from about +20 to about −20 degrees, e.g., from about +15 to about −15, from about +10 to about −10, or even from +5 to about −5 degrees. Thus, the pessary body 102 can have a first height H1 along a first side of the pessary body from the first end 104 to the second end 106, and a second height H2 along a second side of the pessary body 102 from the first end 104 to the second end 106, the first height H1 being greater than the second height H2. However, in alternative embodiments, the first and second planes P₁ and P₂ can be parallel to one another.

The second end 106 includes a second perimeter in a second cross-sectional plane that is perpendicular to the second central axis A₂. The second perimeter can have any suitable shape in the second cross-sectional plane. Further, the second end 106 can have a second area within the second perimeter. The second area can be greater than the first area. Thus, the cross-section of the second end 106 can be larger than the cross-section of the first end 104. The second perimeter can have any suitable shape in the second cross-sectional plane and can have a shape that is the same as or different from that of the second opening 114. For example, the second perimeter can have an oblong shape in the second cross-sectional plane. FIG. 16 shows four alternative examples of pessaries, where each includes a second end that has a different oblong shape. The oblong shape of the second end 106 can prevent the pessary 100 from rotating about the patient's cervix 202 in the vaginal canal 206. Rotation of pessaries around the cervix causes friction which can irritate the cervix. It will be understood that, in alternative embodiments, the second perimeter can have another suitable shape such as a circle.

The pessary body 102 also includes an exterior wall 108 that extends between the first end 104 and the second end 106. For example, the exterior wall 108 can extend from the first end 104 towards the second end 106. In some embodiments, the exterior wall 108 can extend from the first end 104 to the second end 106. The exterior wall 108 has an outer surface 108a and an interior surface 108b opposite the outer surface 108a. The outer surface 108a can have a shape that substantially conforms to the shape of the vaginal canal 206. For example, the outer surface 108a can flare outwards as it extends from the first end 104 towards the second end 106. In at least one example, the outer surface 108a can be substantially frustoconical in shape. The inner surface 108b defines a channel 116 that extends between the first end 104 and the second end 106. The exterior wall 108 can define a closed shape around the channel 116. Thus, the exterior wall 108 can enclose the channel 116. The channel 116 is in fluid communication with the first opening 112. Further, the channel 116 can be in fluid communication with the second opening 114.

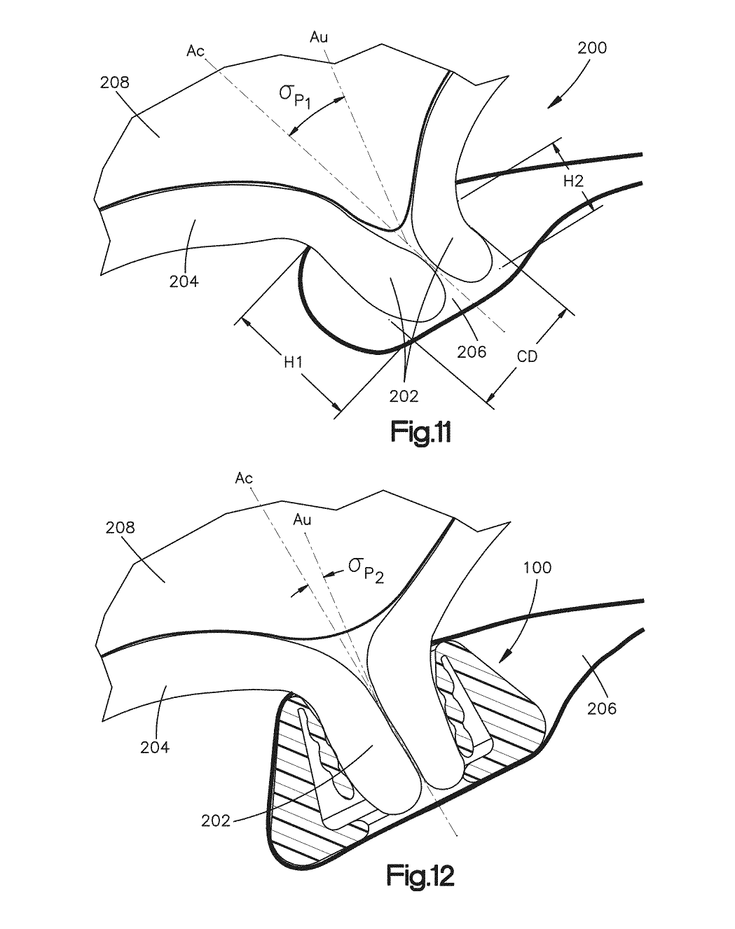

The pessary body 102 can have at least one height, such as heights H1 and H2, from the first end 104 to the second end 106 that is sized such that the first end 104 is biased against the uterine wall 204 when the pessary 100 is supported by the cervix 202 in the vaginal canal 206. The first central axis A₁ of the first opening 112 can intersect the second central axis A₂ of the second opening 114 at a first angle σ_{c}. The first angle σ_{c} may be referred to as a correction or rotation angle. Referring specifically to FIGS. 11 and 12, the correction angle σ_{c} can be selected to reduce an angle between a uterine longitudinal axis A_{U} (defined by a length of the patient's uterus) and a cervical central axis A_{C}. For example, the correction angle σ_{c} can be selected such that the pessary 100 rotates the cervix 202 by the correction angle σ_{c} from an uncorrected cervical angle σₚ₁ between axes A_{U} and A_{C} shown in FIG. 11 to a corrected or reduced cervical angle σₚ₂ between axes A_{U} and A_{C} shown in FIG. 12. Thus, correction angle σ_{c} can be equal to σₚ₁-σₚ₂. In some embodiments, the correction angle σ_{c} can be between −20° to about +20°. Further, the pessary 100 can be designed so as to produce a reduced cervical angle σₚ₂ in a range of about −10° to about +10°. In one specific example, the reduced cervical angle σₚ₂ can be zero degrees, or about zero degrees.

The pessary 100 can further be designed such that, when the pessary 100 is supported by the cervix 202, the first plane P₁ forms an angle with the cervical central axis A_{C} that is between about 80° and about 100°. In some embodiments, the angle may be about 90°. Rotating the cervix 202 can aid in drawing the walls of the cervix 202 closer together. Further, rotating the cervix 202 can reduce the curvature of the amniotic sac 208 at the cervical opening, thereby straightening or flattening out the lower end of the amniotic sac 208. This in turn can reduce the force applied by the amniotic sac 208 to the inner sides of the walls of the cervix 202 that biases the walls of the cervix 202 away from one another.

The interior wall 110 can extend from the first end 104 towards the second end 106 and within the channel 116. Thus, the interior wall 110 can extend inwardly into the channel 116 from the first end 104. The interior wall 110 can be configured as a downwardly extending flange or collar. In at least one embodiment, the interior wall 110 can include a lower free end 110c, and the interior wall can extend from the first end 104 and terminate at the lower free end 110c. The interior wall can have one or more cross-sections having any suitable shape such as any suitable closed shape. In at least one embodiment, the cross-sections can have an annular shape. The one or more cross-sections can decrease in size as the interior wall 110 extends towards the second end 106. The interior wall 110 includes an inner (interface) surface 110a configured to engage the cervix 202 of the patient so as to secure the pessary 100 to the cervix 202. The inner surface 110a can define a second channel 118 that is configured to receive the cervix 202. The interior wall 110 can define a closed shape around the second channel 118. Thus, the interior wall 110 can enclose the second channel 118. The second channel 118 can be in fluid communication with the first opening 112. The second channel 118 can further be in fluid communication with one or both of the channel 116 and the second opening 114. The second channel 118 can have one or more cross-sections having any suitable shape such as any suitable closed shape. In at least one embodiment, the cross-sections can have a circular shape. The one or more cross-sections can decrease in size as the channel 118 extends towards the second end 106. Thus, in some embodiments, the second channel 118 can taper inward as the channel 118 extends towards the second end 106, although alternative embodiments may not taper. For example, in some embodiments, the channel 118 can have a frustoconical shape.

The interior wall 110 can be configured to expand as the cervix 202 is received in the second channel 118. In the expanded state, the interior wall 110 is biased inwardly toward the cervix 202 so as to apply a biasing force on the cervix 202. The inner surface 110a can provide a greater contact area with the cervix 202 than that of conventional pessaries that provide only a single point or line of contact with a cervix. As a result, pessary 100 can exert smaller contact forces on the outside of the cervix 202 than conventional pessaries by more evenly distributing the contact forces along a length of the cervix 202.

The interior wall 110 can include an outer surface 110b opposite the inner surface 110a. The outer surface 110b can face the inner surface 108b of the exterior wall 108. Thus, the pessary body 102 can define a space between the exterior wall 108 and the interior wall 110, and in particular between the outer surface 110b and the inner surface 108b. The space can be an air gap. Further, the space can decrease in size when the interior wall 110 is expanded over the cervix 202.

In some embodiments, the interior wall 110 can define one or more ridges 120 that extend into the second channel 118. The ridges 120 can be annular in shape. The interior wall 110 can additionally or alternatively define at least one recess 122 that extends into the inner surface 110a of the interior wall. The at least one recess can be annular in shape. When pessary 100 is placed around the patient's cervix 202, the cervix 202 may deform over time in order to expand into the at least one recess 122. As described elsewhere herein, the pessary—or at least a portion of the pessary—can be formed of a compliant material. As the patient's cervix expands, the pessary 100 can become more securely attached in place.

A pessary may comprise materials (or material regions) of different compliance. As one example, a pessary may be constructed such that the first end is comparatively compliant so as to facilitate insertion into the patient. The pessary may be constructed so as to be comparatively less compliant in a direction along the cervical axis A_{C} so as to enable the pessary to resist forces related to cervical shortening.

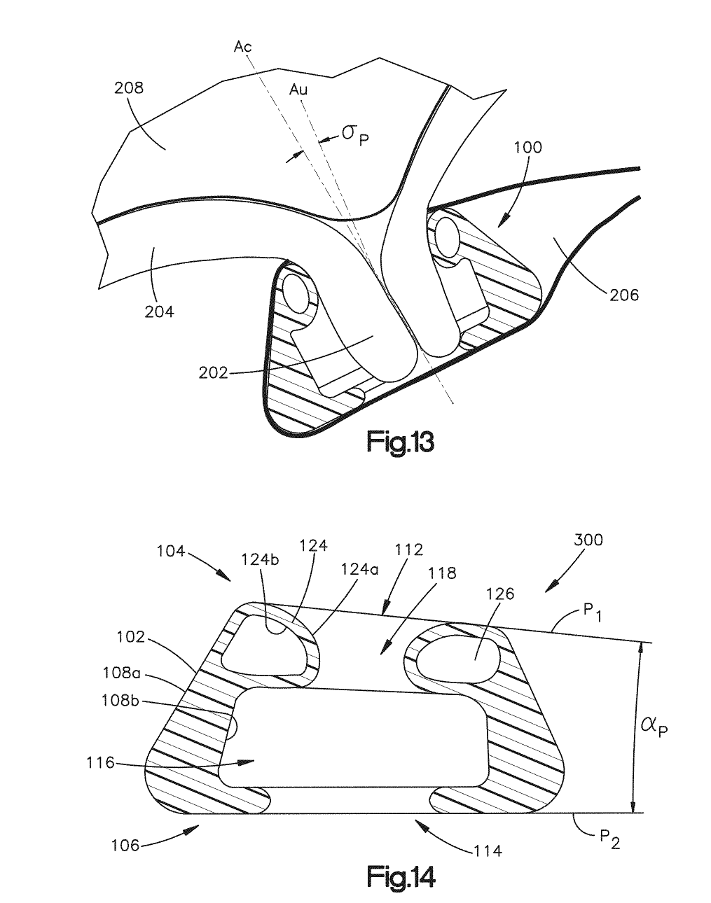

Referring to FIGS. 13 and 14, an alternative embodiment is shown in which like reference numerals identify similar or identical elements to those discussed above. The pessary 300 includes an interior wall 124 that includes an inner surface 124a configured to engage the cervix 202 of the patient so as to secure the pessary 300 to the cervix 202. The inner surface 124a can define a second channel 118 that is configured to receive the cervix 202. The interior wall 124 can define a closed shape around the second channel 118. Thus, the interior wall 124 can enclose the second channel 118. The second channel 118 can be in fluid communication with the first opening 112. The second channel 118 can further be in fluid communication with one or both of the channel 116 and the second opening 114. The inner surface 124a can provide a greater contact area with the cervix 202 than that of conventional pessaries that provide only a single point or line of contact with a cervix. As a result, pessary 300 can exert smaller contact forces on the outside of the cervix 202 than conventional pessaries by more evenly distributing the contact forces along a length of the cervix 202.

The interior wall 124 can include an outer surface 124b opposite the inner surface 124a. The outer surface 124b can face the inner surface 108b of the exterior wall 108. Further, the interior wall 124 can include a first end 124c and a second end 124d. The first and second ends 124c and 124d can be attached to the exterior wall 108 so as to define a closed void 126 between the outer surface 124b and the inner surface 108b. The void 126 can decrease in size when the interior wall 124 is compressed against the cervix 202. The void 126 can be filled with air, liquid, gel, or other suitable material that is compliant/compressible so as to allow the interior wall 110 to compress against a cervix 202.

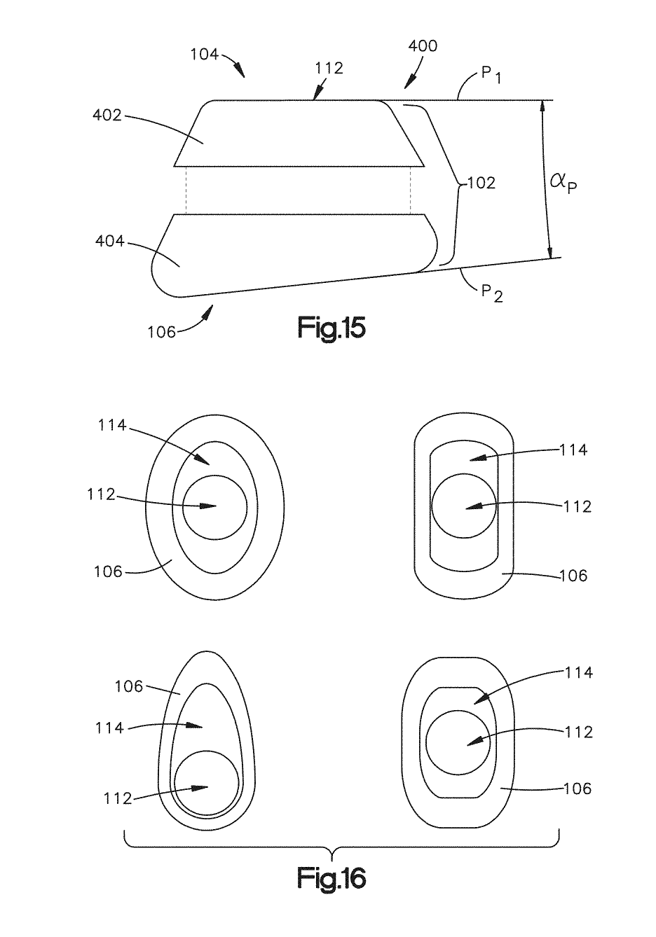

With reference to FIG. 15, in at least one embodiment, the pessary body 102 can include a first portion 402 and a separate second portion 404 that can be coupled to the first portion 402. The first portion 402 can include the first end 104 configured as described above. Further, the second portion 404 can include the second end 106 configured as described above. The first portion 402 and the second portion 202 can be coupled to one another using any suitable coupling technique such as gluing, ultrasonic welding, infrared welding, laser welding, press-fitting, or any other suitable technique. The first portion 402 can be selected or manufactured to have a first opening 112 that is sized for a particular patient's cervix 202. The second portion 404 can be selected or manufactured to have a second end 106 that offset from the first end 104 such that the second plane P2 is offset from the first plane P1 by an angle α_{P} that is specific to a particular patient's anatomy. Further, the first and second portions 402 and 404 can be selected or manufactured to have a height or heights (e.g., H1, H2) that are specific to a particular patient's anatomy. Thus, pessaries of the disclosure can be provided in set arrays or ranges of sizes and shapes and then can be assembled in various combinations depending on the anatomy and needs of a particular patient.

In some embodiments, pessaries of the disclosure can include a coating on pessary body, such as a coating comprising progesterone or other medicaments. Without being bound to any particular theory of operation, such a coating may function similar to a suppository. Further, pessary bodies of the disclosure can be made out of any suitable biocompatible material, including (without limitation) silicone and rubber.

Pessaries of the disclosure can be customized to the specific anatomy, for example based on the patient's anatomical measurements as determined by any combination of the methods described herein, of a patient in order to minimize a likelihood of preterm birth. With reference to FIGS. 11 and 12, a patient-specific pessary can be designed by measuring a patient's anatomy via one or both of a transabdominal ultrasound and a transvaginal ultrasound, and designing a complementary pessary that appropriately tilts the cervix in the correct direction and fits onto the outer cervix while limiting single-points of contact with the cervix. The custom-fit pessary can be designed based on anatomy dimensions such as cervical length, anterior uterocervical angle, external cervical diameter, and height of the vaginal canal. A custom-fit pessary can have: 1) an inner diameter that matches the outer diameter of the cervix, with an interior wall having that contacts the cervix along a surface area rather than at a single point of contact, and 2) at least one height based on the vaginal canal dimensions and cervical canal angle. As such, the pessary can orient the cervix relative to the uterus at a desired angle, and the pessary can be positioned close to the internal orifice (os) of the uterus. The custom-fit pessary can then be validated by running a computational simulation of the pessary geometry placed within a computer model of the patient's anatomy to understand how it will interact within the specific environment.

For instance, a customized pessary can be designed by identifying a uterine longitudinal axis A_{U} (defined by a length of the patient's uterus) and a cervical axis A_{C} (defined by a line along which the patient's cervical opening extends) using ultrasound, though it is not a requirement that a pessary according to the present disclosure be so designed. A first angle σₚ, defined by the angle between the uterine longitudinal axis A_{U} and a cervical axis A_{C} may be measured. Ultrasound may also be used to measure a cervical diameter CD of a patient, the cervical diameter being measured perpendicular, or approximately perpendicular to the cervical axis A_{U}. Additionally, ultrasound may be used to measure at least one height H of a vaginal canal 206 of the patient. For example, ultrasound may be used to measure a posterior height H₁ of the vaginal canal 206 and an anterior height H₂ of the vaginal canal 206.

A method of designing or forming a pessary configured to prevent a preterm birth in a patient can include a step of obtaining a measurement of two or more of the angle σₚ, the cervical diameter CD, and the height H, such as H₁ and/or H₂ from the patient. The method can further include forming the pessary based on two or more of the first angle, the cervical diameter, and the at least one height. The pessary can be designed or formed to include a pessary body having a first end and a second end offset from one another, the first end defining a first opening that is configured to receive the cervix. The pessary can be formed such that the pessary body has an exterior wall that extends from the first end towards the second end, the exterior wall having an outer surface, and an inner surface opposite the outer surface, the inner surface enclosing a channel that extends between the first and second ends and that is in fluid communication with the first opening. The pessary can further be formed to have an interface surface extends between the first end and the second end, the interface surface configured to engage the cervix so as to secure the pessary to the cervix.

For example, the pessary can be designed or formed to have a first opening 112 that is sized based on the cervical diameter CD of the patient. As another example, the pessary can be designed or formed to have the exterior wall 108 that has at least one height that is based on the at least one height H of the vaginal canal 206 of the patient. As yet another example, the pessary can be designed or formed to have one or both of (i) an angle α_{P} between the first and second planes P₁ and P₂ and (ii) an angle σ_{c} between axes A₁ and A₂ that reduces the angle between the uterine longitudinal axis A_{U} and the cervical axis A_{C} (e.g., from angle σₚ₁ in FIG. 11 to angle σₚ₂ in FIG. 12). For example, the pessary can be designed or formed to rotate the cervix 202 to have a reduced angle σₚ₂ in a range of about −10° to about +10°. In one specific example, the reduced cervical angle σₚ₂ can be zero degrees, or about zero degrees.

In some embodiments, the step of obtaining the at least two of the angle σₚ₁, the cervical diameter, and the at least one height can include a step of measuring the at least two of the angle σₚ₁, the cervical diameter, and the at least one height of the patient. Additionally or alternatively to the measuring step above, the obtaining step can comprise receiving the at least two of the first angle, the cervical diameter, and the at least one height at a computing device.

In some embodiments, the step of forming the pessary can include selecting both i) a first portion of the pessary that includes the first end, and ii) a second portion of the pessary that includes the second end, the selecting being based on two or more of the first angle, the cervical diameter, and the at least one height, and the second portion being separate from the first portion. In such embodiments, the step of forming the pessary can further include coupling the first and second portions to one another. In some embodiments, the forming step can comprise generating a computer model of the pessary based on the at least two of the first angle, the cervical diameter, and the at least one height at a computing device. In some such embodiments, the forming step can comprise forming the pessary based on the computer model. Further, in some embodiments, the forming step can comprise forming the pessary using additive manufacturing (e.g., a three-dimensional printer), injection molding, sculpting, or by subtractive manufacturing. The pessary may or may not be formed based on the computer model. Further, pessaries of the disclosure can be coated with, for example, a progesterone coating.

As used herein, the term “oblong” refers to a shape that has an aspect ratio (of longitudinal dimension to lateral dimension) greater than 1:1, e.g., a non-circular shape. Ovoid and trapezoidal shapes are considered “oblong,” for example, though the term “oblong” encompasses other shapes that have an aspect ratio other than 1:1. Without being bound to any particular theory, an oblong-shaped second end 106 may allow the pessary to resist being turned, twisted, or being otherwise moved out of a desired position in the patient during the patient's activities.

In some embodiments, the first end 104 can have a first cross-sectional dimension along a select direction, and the second end can have a second cross-sectional dimension along the select direction. The second cross-sectional dimension can be greater than the first cross-sectional dimension. For example, a width of the second end 106 can be greater than the diameter of the first end 104.

In some embodiments, the second end 106 is in fluid communication with the first end 104, and the exterior wall 108 defines an interior volume that is eccentric (e.g., a revolved trapezoid) in configuration. In other embodiments, the second end 106 is in fluid communication with the first end 104 and the exterior wall 108 defines an interior volume that is cylindrical or substantially cylindrical in shape.

Also provided are methods of predicting a likelihood of preterm birth in a subject, the methods comprising: generating a computer simulation of a mechanical environment of pregnancy from a series of maternal anatomical measurements, the maternal anatomical measurements being derived from a series of ultrasound-based images; and predicting a likelihood of preterm birth if one or more factors characteristic of increased risk of preterm birth are present in the computer simulation.

Methods of preventing preterm birth in a subject are also provided. The methods comprise administering any of the pessaries disclosed herein to the subject, wherein one or more factors characteristic of increased risk of preterm birth are present in a computer simulation of a mechanical environment of pregnancy. The computer simulation of a mechanical environment of pregnancy can be generated from a series of maternal anatomical measurements derived from a series of ultrasound-based images.

The use of a computer simulation of a mechanical environment of pregnancy for preventing preterm birth in a subject are provided. The use comprises predicting a likelihood of preterm birth in the subject if one or more factors characteristic of increased risk of preterm birth are present in the computer simulation, wherein the computer simulations are derived from a series of ultrasound-based images, and one or more factors characteristic of increased risk of preterm birth are present, administering any of the pessaries disclosed herein to the subject.

Factors characteristic of increased risk of preterm birth include, e.g., a short cervix, which characteristic is presently used as a primary indicator of risk of preterm birth. (In some instances, the short cervix is less than 25 mm in length.) Other factors considered in current practice include prior history of preterm birth, a multiple pregnancy (twins, triplets, etc.), positive cervicovaginal fetal fibronectin, and certain lifestyle habits. As disclosed herein, however, a variety of other, previously-unconsidered factors may be evaluated, including, e.g., increased cervical angle, large offset of the cervical inner canal from the main longitudinal axis of the uterus, and inadequate fiber stiffness. As described in, e.g., paragraphs [0116] and [0117] and in Table 1 herein, various measurements may be used to assess these further factors.

The maternal anatomical measurements being derived from a series of ultrasound-based images can include one or more of placenta location, placenta volume, fetal biometrics, amniotic fluid index, uterine diameter, uterine thickness, cervical length, cervical diameter, cervical angle with anterior lower uterine segment, cervical os, mechanical load, and tissue stretch.

Further provided are methods of predicting an amount of cervical stretch likely to occur in a subject during pregnancy, the method comprising:

performing ultrasound-based imaging of the subject to obtain a series of maternal anatomical measurements;

generating a computer simulation of a mechanical environment of pregnancy from the series of maternal anatomical measurements, the computer simulation representing a base-line for the patient;

applying a intrauterine pressure to the computer simulation of the mechanical environment; and

predicting the amount of cervical stretch that would result from the intrauterine pressure.

Methods of preventing cervical stretch in a subject during pregnancy are also provided. The methods comprise administering any of the pessaries disclosed herein to the subject, wherein, upon application of an interuterine pressure to a computer simulation of a mechanical environment of pregnancy, the computer simulation indicates that cervical stretch is likely to occur. The computer simulation of a mechanical environment of pregnancy can be generated from a series of maternal anatomical measurements derived from a series of ultrasound-based images. The computer simulation can represent a base-line for the subject.

The use of a computer simulation of a mechanical environment of pregnancy for preventing cervical stretch in a subject during pregnancy are also provided. The use comprises applying an intrauterine pressure to the computer simulation of the mechanical environment of pregnancy and predicting the amount of cervical stretch that would result from the intrauterine pressure, and, if cervical stretch is likely to occur, administering any of the pessaries disclosed herein to the subject. The computer simulation of the mechanical environment of pregnancy can be generated from a series of maternal anatomical measurements derived from ultrasound-based imaging of the subject. The computer simulation can represent a base-line for the subject.

The series of maternal anatomical measurements can comprise one or more of placenta location, placenta volume, fetal biometrics, amniotic fluid index, uterine diameter, uterine thickness, cervical length, cervical diameter, cervical angle with anterior lower uterine segment, cervical os, mechanical load, and tissue stretch. Also disclosed herein are computer simulations of a mechanical environment of pregnancy comprising: a memory adapted to store computer instructions; a database; and a processor adapted to process the computer instructions to implement a computer simulation of a mechanical environment of pregnancy, wherein the computer simulation of a mechanical environment of pregnancy comprises one or more measurements of a longitudinal uterine diameter, an anterior-posterior diameter, a cervical os offset from the longitudinal diameter, a transverse uterine diameter, a uterine wall thicknesses, a cervical length, a cervical diameter, a canal width, an angle with the anterior lower uterine segment, and an angle with periosteum of the symphysis pubis.

In some embodiments, the computer simulation can be used in predicting a likelihood of preterm birth in a subject, the predicting comprising determining if one or more factors characteristic of increased risk of preterm birth are present in the computer simulation. Suitable factors characteristic of increased risk of preterm birth comprise a short cervix. In some aspects, the short cervix is less than 25 mm in length.

In some embodiments, the computer simulation can be used in predicting if cervical stretch is likely to occur in a subject during pregnancy, comprising applying an intrauterine pressure to the computer simulation and predicting the amount of cervical stretch that would result from the intrauterine pressure.

In the disclosed computer simulations, the one or more measurements are derived from placenta location, placenta volume, fetal biometrics, amniotic fluid index, uterine diameter, uterine thickness, cervical length, cervical diameter, cervical angle with anterior lower uterine segment, cervical os, mechanical load, and tissue stretch.

### EXAMPLES

The following examples are provided to further describe some of the embodiments disclosed herein. The examples are intended to illustrate, not to limit, the disclosed embodiments.

A baseline model at 25 weeks of gestation was characterized, and to visualize the impact of cervical structural parameters on tissue stretch we evaluated the model sensitivity to: (1) anterior uterocervical angle, (2) cervical length, (3) posterior cervical offset, and (4) cervical stiffness. We found that cervical tissue stretching is minimal when the cervical canal is aligned with the longitudinal uterine axis and a softer cervix is more sensitive to changes in the geometric variables tested.

To address the need for a fast, flexible, and affordable computational assessment of the soft tissue mechanics in pregnancy, a parametric finite element model that utilized ultrasound images of a pregnant abdomen at 25 weeks gestation was built. A user-friendly routine to convert these ultrasound parameters into a CAD model of the pregnant anatomy was scripted. This CAD model was used in finite element simulations to calculate the distribution of tissue stress and stretch at 25 weeks of gestation, where material parameters and loading and boundary conditions were informed or inferred by previously reported studies. The effect of cervical structural parameters on the magnitude of tissue stretch at the cervical opening to the uterus (i.e. cervical internal os), which is the anatomical site of clinically-observed cervical failure, were investigated.

### Ultrasound Measurements of Maternal Anatomy

Geometric dimensions of the uterus, cervix, and their position in reference to the symphysis pubis as a bony reference landmark were taken via ultrasound (GE Voluson E8) (FIGS. 3 and 4). All measurements were taken transabdominally or transperineally using the transabdominal probe (GE RAB4-8D, real-time 4D volume, curved array transducer, 4-8.5 MHZ). For the baseline model, dimensions were measured from a 35-year-old patient with no prior pregnancies at 25 weeks gestation with an empty bladder. The patient subsequently delivered the neonate at 40 weeks.

Uterine diameters (UDs) were measured with the extended view imaging feature of the Voluson E8, which automatically registered adjacent ultrasound images as the probe was swept across the abdomen from the fundus to the pubic bone at a steady rate of 2 cm/s. With this sagittal view, measurements of uterus longitudinal diameter (UD1), anterior-posterior diameter (UD2+UD3), and the offset of the cervical internal os from the uterus longitudinal diameter (PCO) were obtained (FIG. 3, (A)). To measure the transverse uterine diameter (UD4) in an extended axial view, the transabdominal probe was swept from left to right across the midabdomen and the uterus measured at its widest point (FIG. 3, (B)). Uterine wall thicknesses (UT1-5) were measured at multiple locations from the fundus to the lower uterine segment (LUS) with the transabdominal probe in a standard clinical resolution (FIGS. 3, (C) and 4 (A)), and were considered the echogenic signal from the serosa to the decidua. Cervical length (CL), diameter (CD1), canal width (CD2), angle with the anterior LUS (AUCA), and angle with periosteum of the symphysis pubis (CA1) were assessed via transperineal scans (FIG. 4, (B) and (C)).

### Computer (CAD) Model of Pregnancy

The maternal ultrasonic parameters were converted into CAD geometries with a custom computer script (Trelis Pro 15.1.3, csimsoft LLC). Geometries of the uterus, cervix, fetal membranes, vaginal canal, and abdomen were created with Boolean addition and subtraction of geometric primitives (FIG. 1 (A)). Dimensions for the baseline model are provided in Table 1. For this initial model, the uterus was built by transforming two spherical shells into ellipsoids. The interior uterus was scaled to the diameters obtained during ultrasound (UD1-4) and rotated in relation to the reference angle of the symphysis pubis (CA1). The current iteration of this model does not have CA1 as a measured value from the patient. Instead, it uses an arbitrary value of 15°. The outer shell was then scaled, translated, and rotated to accommodate differences in uterine wall thickness (UT1-5) in the anterior-posterior, superior-inferior, and left-right directions.

TABLE 1
Baseline ultrasound measurements

| Dimension | Measured Value |
|---|---|
| UD1 | 192 mm |
| UD2 | 68 mm |
| UD3 | 55 mm |
| PCO | 25 mm |
| UD4 | 215 mm |
| UT1 | 5 mm |
| UT2 | 6 mm |
| UT3 | 6 mm |
| UT4 | 6 mm |
| UT5 | 5 mm |
| AUCA | 90° |
| CA1 | 15°* |
| CL | 30 mm |
| CD1 | 30 mm |
| CD2 | 4 mm |

UD = uterine diameter,
PCO = posterior cervical offset,
UT = uterine thickness,
AUCA = anterior uterocervical angle,
CA = cervical angle.
*Arbitrary value, not measured value, CL = cervical length, CD = cervical diameter

The cervix was built by creating a cylinder representing the diameter of the inner canal (CD2) and subtracting that volume from a larger cylinder representing the outer cervical diameter (CD1) and cervical length (CL). The resultant hollow cylinder was then moved and rotated according to posterior cervical offset (PCO) and anterior cervical angle (AUCA). The cylinder was rounded at its corners to match the anatomical rounding of the uterocervical junction, and to replicate the roundness of the most exterior end of the cervix (i.e. external os).

For purpose of tissue loading analysis, the cervix was then separated into three different regions: an upper portion, a lower portion, and the internal os region (FIG. 1 (A)). First, the cylindrical representation of the cervix was cut by a plane normal to the external os at a fixed distance of 15 mm from the internal os. Second, the top portion of the cervix was then separated by a surface extended from a smaller cylinder with a diameter that was twice of the cervical inner canal. Lastly, the vaginal canal was built by fitting a spline to three vertices located at the outside edges of the external os and one vertex at the approximate location of the vaginal introitus and the fetal membrane was generated with uniform thickness based on the contours of the inner uterine wall.

### Finite Element Mesh Generation

All meshes were generated using the automatic and manual meshing tools in Trelis Pro (v15.1.3, csimsoft LLC). The fetal membranes were meshed with hexahedral elements, while all other volumes were meshed with tetrahedral elements. Mesh properties varied from model to model. The baseline model mesh is provided in Table 2. All volumes except the fetal membranes were meshed with 10-node tetrahedral elements. The fetal membranes were meshed as a single continuous layer of hexahedral elements with thickness of 0.1 mm and no edges longer than 3 mm. The uterus and cervix were connected at the node level to one another, so their boundaries were shared and moved congruently. Where the uterus and cervix shared a boundary with the abdomen volume, those boundaries were also node-tied. The lower cervix was not tied to the interior vaginal canal, but floated freely inside the vaginal fornix.

The mesh density of the cervix was set to the finest setting by the inherent Trelis element density function, in order to yield the most accurate deformation results for the analysis.

TABLE 2
Mesh properties for baseline model

| | | | | | | | Internal |
|---|---|---|---|---|---|---|---|
| | | | | | Upper | Lower | Os |
| | Total | Uterus | Membrane | Abdomen | Cervix | Cervix | Region |
| Element | — | Tet | Hex | Tet | Tet | Tet | Tet |
| Type | | | | | | | |
| Element | 180,735 | 35,246 | 9,600 | 39,933 | 51,239 | 27,422 | 17,295 |
| Count | | | | | | | |
| Average | — | 16.3 mm³ | 0.994 mm³ | 613 mm³ | 0.236 mm³ | 0.344 mm³ | 0.131 mm³ |
| Element | | | | | | | |
| Volume | | | | | | | |

### Material Properties

The cervix and uterus materials were treated as continuously distributed fiber composites with a compressible neo-Hookean ground substance. This hyperelastic solid model was developed to describe the tension-compression nonlinearity in human and mouse cervical tissue. Considering not much is known about the multi-axial material behavior of these tissues during pregnancy, the full range of possible properties were investigated, where term pregnant (PG) tissue was considered the remodeled tissue and non-pregnant (NP) tissue represented the not remodeled tissue.

The total Helmholtz free energy density Ψ^{TOT} for the uterine and cervical materials were given by Formula (1):

Ψ^{TOT}(F)=Ψ^{GS}(F)+Ψ^{COL}(F), (1)

where F is the deformation gradient. The free energy density of the ground substance Ψ^{GS} is given by a standard isotropic, compressible neo-Hookean relation as shown in Formula (2):

$${Ψ}^{GS} = \frac{μ}{2} ( {I}_{1} - 3 ) - μ ln J + \frac{λ}{2} {( ln J )}^{2} , \qquad ( 2 )$$

where I₁=trC is the first invariant of the right Cauchy-Green tensor C=(F)^{T}F and J=detF is the Jacobian. μ and λ are the standard lame’ constants. These lame’ constants combine to form the Young's modulus and Poisson's ratio of the ground substance

$${E}^{GS} = \frac{μ ( 3 + \frac{2 μ}{λ} )}{1 + \frac{μ}{λ}} and {v}^{GS} = \frac{1}{2 ( 1 + \frac{μ}{λ} )} ,$$

respectively. The strain energy density for the continuously distributed collagen fiber network is given by Formula (3):

$${Ψ}^{COL} = \frac{1}{4 π} {\int }_{0}^{2 π} {\int }_{0}^{π} H ( {I}_{n} - 1 ) {Ψ}_{r}^{fiber} ( {I}_{n} ) sin φ d φ d θ \qquad ( 3 )$$

where the heaviside step function H ensures fibers hold only tension, [θ, φ] are the polar and azimuthal angles in a spherical coordinate system. Iₙ=nₒ·C·nₒ is the square of the fiber stretch, where nₒ=cos θ sin φe₁+sin θ sin φe₂+cos φe₃ in a local Cartesian basis {e₁, e₂, e₃}. Ψ^{fiber} is the strain energy density of a collagen fiber bundle given by Formula (4),

$${Ψ}^{fiber} = \frac{ξ}{β} {( {I}_{n} - 1 )}^{β} \qquad ( 4 )$$

where ξ represents the collagen fiber stiffness with units of stress and β>2 is the dimensionless parameter that controls the shape of the fiber bundle stiffness curve (here, the fiber strain energy density is cast in a different form than the model presented for the human cervical tissue (described in Myers, K. M., et al., 2015. “A continuous fiber distribution material model for human cervical tissue.” J Biomech, 48(9), June, pp. 1533-1540), hence direct comparison can be made by considering the 1/β prefactor here).

To focus this study on the model sensitivity to maternal anatomy and collagen fiber stiffness parameters and not on the collagen ultrastructure, ground substance, or time-dependent properties, simplifying adjustments were made. Both material model fits were conducted on the material behavior after the transient force relaxation response died away. In this present study, a randomly distributed collagen fiber network was used, as opposed to a preferentially-aligned collagen fiber network as presented in Myers, K. M., et al., 2015. J Biomech, 48(9), June, pp. 1533-1540. Cervical material properties used in this study (Table 3) represent collagen fiber parameters fit to the nonpregnant and term pregnant human uniaxial tension-compression data reported in Myers, K. M., et al., 2015. J Biomech, 48(9), June, pp. 1533-1540; Myers, K., et al., 2008. “Mechanical and biochemical properties of human cervical tissue”. Acta Biomater, 4(1), January, pp. 104-116; and Myers, K., et al., 2010. “A study of the anisotropy and tension/compression behavior of human cervical tissue”. J Biomech Eng, 132(2), February, p. 021003, with the ground substance material properties kept constant. Considering the material properties for the cervix at 25 weeks are unknown, the interim cervical fiber stiffness was approximated at constant increments between the known values. Uterine material properties represent a material model fit to passive, nonpregnant and term pregnant human uniaxial tension data reported in Conrad, J. T., et al., 1966. “Passive stretch relationships in human uterine muscle”. Am J Obstet Gynecol, 96(8), December, pp. 1055-1059. Fibers in both the uterus and cervix were randomly distributed. They rotate and stretch in the direction of principle stress. Previous work compared the difference between preferential and randomly distributed fiber directionality in an initial finite element model of pregnancy and found a negligible difference between the two scenarios (See, Fernandez, M., et al., 2015. “Investigating the mechanical function of the cervix during pregnancy using finite element models derived from high-resolution 3D MRI.”. Comput Methods Biomech Biomed Engin, May, pp. 1-14).

TABLE 3
Uterine and cervical tissue variables taken from material fits to
experimental data

| Tissue Description | E^{GS} [kPa] | v^{GS} | β | ξ [kPa] |
|---|---|---|---|---|
| Uterus - remodeled | 2 | 0.3 | 2.71 | 190 |
| Uterus - not | 2 | 0.3 | 3 | 199 |
| remodeled | | | | |
| Cervix - remodeled | 2 | 0.3 | 3.12 | 1.71 |
| Cervix - interim 1 | 2 | 0.3 | 3.12 | 7.89 |
| Cervix - interim 2 | 2 | 0.3 | 3.12 | 36.3 |
| Cervix - interim 3 | 2 | 0.3 | 3.12 | 167 |
| Cervix - not | 2 | 0.3 | 3.12 | 769 |
| remodeled | | | | |

These values are implemented in a continuous fiber distribution material model used in FEBio 2.4.1.

The outer abdomen was treated as a soft nearly incompressible neo-Hookean material with a modulus of 5 kPa. An incompressible Ogden material model based on equibiaxial tensile loading of human amnion (as described in Buerzle, W., and Mazza, E., 2013. “On the deformation behavior of human amnion.”. J Biomech, 46(11), July, pp. 1777-1783) was employed for the fetal membrane layer material properties (Table 4), where the particular form of the Ogden strain energy density, as defined in FeBio, was given by Formula (5):

$${Ψ}^{FM} = {\sum }_{i = 1}^{3} \frac{{c}_{i}}{{m}_{i}^{2}} ( {λ}_{1}^{{m}_{i}} + {λ}_{2}^{{m}_{i}} + {λ}_{3}^{{m}_{i}} - 3 ) . \qquad ( 5 )$$

TABLE 4
Fetal membrane (FM) material properties described
by an Ogden material model in equation 5

| Tissue | c1 | c2 | c3 | | | |
|---|---|---|---|---|---|---|
| Description | [MPa] | [MPa] | [MPa] | m1 | m2 | m3 |
| FM | 0.859 | 0.004 | 0.756 | 27.21 | 27.21 | −16.64 |

### Boundary Conditions and Loading

Boundary conditions were applied as described in FIG. 1 (A). The abdomen was fixed in the x, y, and z directions on its outside surface. The fetal membranes were prescribed a no-slip, tied contact along its outer surface to the inner surface of the uterus and to the inner surface of the upper cervix region. A frictionless sliding contact condition was assigned between the outer surface of the fetal membranes and the internal os region. Anatomically, in normal pregnancy, the outer layer of the fetal membranes are adhered fully to the uterine wall and the upper cervix throughout gestation, and detaches at the onset of labor.

A pressure was applied to the inner surface of the fetal membranes to represent the intrauterine pressure (IUP). IUP magnitude was informed by previous studies that measured the amniotic sac cavity pressure via catheter puncture of the fetal membranes, where the values were reported after subtracting the gravitational pressure head from the reading (as described in Fisk, N. M., et al., 1992. “Normal amniotic pressure throughout gestation.” BJOG, 99(1), January, pp. 18-22). IUP at 25 weeks (0.817 kPa) was calculated using Formula (6) from Fisk et al.:

ln(y+1)=0.12+0.23x−0.010x²+0.00015x³, (6)

where y is the amniotic pressure in mmHg and x is the gestation in weeks. To compare stretch patterns between models, the pressure was ramped to the value of 40 weeks (2.33 kPa) and to the value of a labor contraction (8.67 kPa) (as described in Buhimschi, C. S., et al., 2004. “Intrauterine pressure during the second stage of labor in obese women.” Obstet Gynecol, 103(2), February, pp. 225-230).

### Finite Element (FE) Analysis and Evaluation

FE analyses were performed in FEBio 2.4.2 (http://www.febio.org.). Stress and stretch data were plotted as a function of IUP in PostView (PostView 1.19.1), FEBio's post-processor for visualization and analysis. These data were then imported into MATLAB (MATLABR2014a) for further post analysis. To describe the deformation of the cervix, the extent of cervical 1st principal right stretch was evaluated as a percentage of the cervical internal os volume (FIG. 1 (A)) above a 1.05 stretch threshold. The right stretch in this context is the symmetric tensor U in the polar decomposition of the deformation gradient F=RU.

### Sensitivity to Cervical Structural Parameters

After the evaluation of the baseline model, cervical structural parameters were scaled individually in order to assess each variable's impact on cervical internal os stretch. The range of values were based on literature values (as described in Dziadosz, M., et al., 2016. “Uterocervical angle: a novel ultrasound screening tool to predict spontaneous preterm birth.”. Am J Obstet Gynecol; Prado, C. A. d. C., et al., 2016. “Predicting success of labor induction in singleton term pregnancies by combining maternal and ultrasound variables.”. J Matern Fetal Neonatal Med, January, pp. 1-35) and represented clinical significance. These parameters were: anterior uterocervical angle (AUCA), cervical length (CL), posterior cervical offset (PCO), and cervical stiffness (Table 5). Stretch magnitude and distribution were compared at a contraction-level IUP of 8.67 kPa to illuminate patterns. AUCA in this analysis was defined as the angle between the cervical inner canal and the anterior LUS (FIG. 4, (B)).

TABLE 5
Model geometries, with ranges for each varied parameter.
AUCA = anterior uterocervical angle, CL = cervical
length, PCO = posterior cervical offset

| | Cervical | Cervical | | |
|---|---|---|---|---|
| | Angle | Length | Cervical Offset | Cervical Fiber |
| Model | (°) | (mm) | (mm) | Stiffness ξ (kPa) |
| Baseline | 90 | 30 | 25 | 1.71 & 867 |
| AUCA | 90→110 | 30 | 25 | 1.71 & 867 |
| CL | 90 | 25→40 | 25 | 1.71 & 867 |
| PCO | 90 | 30 | 0→25 | 1.71 & 867 |
| Cervical | 90 | 30 | 25 | 1.71 → 867 |
| stiffness | | | | |

AUCA in this analysis was defined as the angle between the cervical inner canal and the anterior LUS (FIG. 4, (B)). AUCA was varied in ten degree increments from 90° in the baseline model to the most extreme value of 110° with respect to the anterior LUS. CL was varied in 5 mm increments from 25 mm (a clinical short cervix) to 40 mm. CL in this analysis was defined as the length of the inner canal from the internal os to the external os (FIG. 4, (B)). PCO was varied in 5 mm increments from 0 mm to the baseline value of 25 mm. PCO in this analysis was defined as the distance from the longest uterine diameter to the cervical internal os (FIG. 3, (A)). Cervical stiffness was varied by decreasing fiber stiffness from the NP value of =769 kPa in even increments to reach the PG value of =1.71 kPa. All other parameters in the material model were kept constant.

### Results

Baseline Model—

The baseline pregnancy model at 25 weeks of gestation shows minimal deformation under amniotic sac cavity pressure estimated for that week (Formula 6, IUP=0.817 kPa). At this level of pressure, the maximum level of tensile stretch for the cervix, uterus, and fetal membranes reached 1.04, 1.05, and 1.06 respectively, and the maximum level of compressive stretch for the cervix was 0.86. Minimal tissue stretch at this stage of pregnancy was supported by previous x-ray and histologic studies, where evidence shows that the uterus undergoes dramatic growth with limited stretching in the first half of pregnancy to accommodate the fetus and amniotic fluid (as described in Gillespie, E. C., 1950. “Principles of uterine growth in pregnancy.” Am J Obstet Gynecol, 59(5), May, pp. 949-959). Analysis was done for both remodeled (PG) and not remodeled (NP) uterine and cervical tissue properties for baseline IUP at 25 weeks, baseline IUP at 40 weeks, and contraction-magnitude IUP (Table 5).

To illuminate the pattern of tissue stretch, the model was investigated under a contraction-level intrauterine pressure of 8.67 kPa. There was a jump in tissue stretch distribution at the boundary of the uterus and cervix because of material property definition. Overall, the highest amounts of stretch were located near this uterocervical boundary for the uterus and at the internal os of the cervix. Because of the ellipsoidal shape of the uterus and the placement of the cervix in relation to the uterine axis, tissue stretch patterns followed anatomic quadrants. Zones of high stretch were apparent in the anterior-posterior sections of the uterus and the left-right sections of the cervix (color map not shown). For the uterus the maximum stretch was directed along the meridian in the anterior and posterior quadrants and along the circumference in the left-right quadrants. Throughout the uterine thickness, the stretch was at a maximum on its inner surface and decreased towards the outer surface. These stretch concentration patterns may vary for differing uterine shapes and sizes.

TABLE 6
1st principal right stretch for the baseline geometry

| | Cervix and uterus material properties | | |
|---|---|---|---|
| | | Not remodeled | remodeled |
| Intrauterine | 0.817 kPa | 0% | 0% |
| pressure (IUP) | 2.33 kPa | 0.4% | 8.2% |
| | 8.67 kPa | 25.3% | 92.6% |

1st principal right stretch for the baseline geometry evaluated with IUPs of (A) 25 weeks = 0.817 kPa, (B) 40 week = 2.33 kPa, and (C) contraction = 8.67 kPa. The percentage reports the volume fraction of the cervical internal os region above a 1.05 stretch.

For the upper part of the cervix, its outer edges are dictated by the direction of the uterine wall tension, where the anterior-posterior cervix is pulled in a radial direction and the left-right quadrants are pulled in circumferential tension. The stretch pattern of the inner core of the cervix did not show quadrant patterns. Instead, the first principal stretch was directed circumferentially in all anatomic quadrants (color maps not shown). For compressive stretch, the distribution was off-centered and the maximum magnitude was located in the posterior section (color maps not shown). Second principal stretch was largest in the left and right quadrants of the uterus (color maps not shown), and maximum shear strain occurred at the posterior uterus and uterocervical interface (color maps not shown). These stretch patterns were most likely dominated by the geometric features of the uterus and fetal membranes adhesion at both the inner uterine surface and the inner surface of the upper cervix region. The stretch provided here is for a uniform intrauterine pressure and does not include the fluid pressure head due to gravity. Gravitational forces will most likely shift this distribution towards the anterior direction.

Cervical Structural Parameters—

Both the geometric and material properties of the cervix influence the distribution and magnitude of tissue stretch. Results indicated that geometric variations in anterior uterocervical angle (AUCA), cervical length (CL), and posterior cervical offset (PCO) are all more influential in a softer cervix (Table 7). The geometric parameter PCO had the largest effect on the loading at the internal os for both the soft pregnant (PG) cervix and the stiff nonpregnant (NP) cervix. Aligning the cervical canal with the uterine longitudinal axis reduced the amount of tissue stretch at the internal os. For the PG cervix, when PCO=0 mm the volume fraction of cervical tissue above the 1.05 stretch threshold reduced by 50% compared to the most extreme case investigated, which was the baseline value of PCO=25 mm. Even for the NP cervix, aligning the cervical canal with the uterine axis reduced the tissue stretch volume fraction by 16%. For the NP cervix,CL and AUCA had a negligible influence on the outcome tissue stretch measurement. For the PG cervix, lengthening the cervix from 25 mm to 40 mm resulted in a 22% reduction in the volume fraction of the cervical internal os above the 1.05 stretch threshold, while varying cervical angle still had little effect on the volume fraction. There were no scenarios in which an NP cervix had the same amount of loading as the PG cervix.

TABLE 7
Summary of results for volume fraction of cervical internal os above a 1.05 stretch threshold

| | | AUCA [°] | | | CL [mm] | | | | PCO [mm] | | | | | |
|---|---|---|---|---|---|---|---|---|---|---|---|---|---|---|
| | Baseline | 90 | 100 | 110 | 25 | 30 | 35 | 40 | 0 | 5 | 10 | 15 | 20 | 25 |
| NP Cervix | 25.3 | 25.3 | 26.0 | 26.1 | 26.2 | 25.3 | 26.0 | 26.8 | 21.2 | 21.2 | 22.0 | 23.3 | 24.1 | 25.3 |
| (stiff) | | | | | | | | | | | | | | |
| volume | | | | | | | | | | | | | | |
| fraction | | | | | | | | | | | | | | |
| above 1.05 | | | | | | | | | | | | | | |
| stretch | | | | | | | | | | | | | | |
| PG Cervix | 92.6 | 92.6 | 94.2 | 94.8 | 93.2 | 92.6 | 80.2 | 72.5 | 46.0 | 48.5 | 50.1 | 58.1 | 82.2 | 92.6 |
| (soft) | | | | | | | | | | | | | | |
| volume | | | | | | | | | | | | | | |
| fraction | | | | | | | | | | | | | | |
| above 1.05 | | | | | | | | | | | | | | |
| stretch | | | | | | | | | | | | | | |

AUCA = anterior uterocervical angle,
CL = cervical length,
PCO = posterior cervical offset. The geometric parameter PCO had the largest influence on the amount of tissue stretch at the cervical internal os, for both a soft PG cervix and a stiffer NP cervix. The most drastic reduction in cervical tissue stretch occurs for a soft cervix that is aligned with the uterine longitudinal axis compared to a 25 mm PCO.

Sensitivity to Anterior Uterocervical Angle (AUCA)—

As anterior uterocervical angle (AUCA) increased and the external os of the cervix was tilted towards the posterior, cervical stretch of the internal os region increased and the distribution of stretch moved posteriorly (color maps not shown). As shown in Table 8, in the not remodeled cervix material model, the 110° AUCA experienced a 3.2% increase in the volume fraction of cervical tissue above the 1.05 stretch threshold from the 90° AUCA. In the remodeled cervix material model, the 110° AUCA experienced an 2.4% increase in the volume fraction from the 90° AUCA. In both models, cervical tissue stretch was minimized when the cervix was aligned with the longitudinal uterine axis (AUCA=90°).

TABLE 8
Uterine and cervical stretch patterns as AUCA was varied

• Cervical collagen fiber
stiffness ξ

| | | 769 kPa | 1.71 kPa |
|---|---|---|---|
| Anterior uterocervical | 90° | 25.3% | 92.6% |
| angle (AUCA) | 100° | 26.0% | 94.2% |
| | 110° | 26.1% | 94.8% |

1st principal right stretch for non-remodeled cervix with AUCA of 90°, 100°, and 110°, and remodeled cervix with AUCA of 90°, 100°, and 110°. The percentage reports the volume fraction of the cervical internal os region above a 1.05 stretch.

Sensitivity to Cervical Length (CL)—

Cervical length (CL) had an influence on cervical loading patterns only when the cervix had remodeled material properties (color maps not shown). As shown in Table 9, when the cervix had not remodeled and was as stiff as the nonpregnant state, increasing the cervical length did not alter the loading pattern at the internal os. For a cervix that was remodeled and was as soft as the term tissue, as cervical length was decreased the stretch at the internal os increased. The 25 mm CL experienced a 28.6% increase in the volume fraction of tissue over 1.05 stretch from the 40 mm CL.

TABLE 9
Uterine and cervical stretch patterns as CL is varied

• Cervical collagen fiber
stiffness ξ

| | | 769 kPa | 1.71 kPa |
|---|---|---|---|
| Cervical length | 25 mm | 26.2% | 93.2% |
| (CL) | 30 mm | 25.3% | 92.6% |
| | 35 mm | 26.0% | 80.2% |
| | 40 mm | 26.8% | 72.5% |

1st principal right stretch for not remodeled cervix with CL of 25 mm, 30 mm, 35 mm, and 40 mm, and remodeled cervix with CL of 25 mm, 30 mm, 35 mm, and 40 mm. The percentage reports the volume fraction of the cervical internal os region above a 1.05 stretch.

Sensitivity to Posterior Cervical Offset (PCO)—

As posterior cervical offset (PCO) increased, cervical stretch of the internal os region increased (color maps not shown). As shown in Table 10, in the not remodeled cervix material model, the 25 mm PCO experienced a 19.3% increase in the volume fraction of tissue over 1.05 stretch from the 0 mm offset, with intermediary values corresponding to this increasing trend. In the PG cervix material model, the 25 mm PCO experienced a 101.3% increase in the volume fraction of tissue over 1.05 stretch from the 0 mm offset, with intermediary values corresponding to this increasing trend. As seen with the CL parameters, the softer cervix was more sensitive to changes in geometric variables.

TABLE 10
Uterine and cervical stretch patterns as PCO (shown top left) is varied

• Cervical collagen
fiber stiffness ξ

| | | 769 kPa | 1.71 kPa |
|---|---|---|---|
| Posterior cervical | 0 mm | 21.2% | 46.0% |
| offset (PCO) | 5 mm | 21.2% | 48.5% |
| | 10 mm | 22.0% | 50.1% |
| | 15 mm | 23.3% | 58.1% |
| | 20 mm | 24.1% | 82.2% |
| | 25 mm | 25.3% | 92.6% |

1st principal right stretch for not remodeled cervix with PCO of 0 mm, 5 mm, 10 mm, 15 mm, 20 mm, and 25 mm, and remodeled cervix with PCO of 0 mm, 5 mm, 10 mm, 15 mm, 20 mm, and 25 mm. The percentage reports the volume fraction of the cervical internal os region above a 1.05 stretch.

Sensitivity to Cervical Stiffness—

As the cervix was made softer by decreasing the value of the collagen fiber stiffness parameters cervical stretch of the internal os region increased (color maps not shown). Keeping all other material parameters the same, the collagen fiber stiffness associated with a remodeled cervix experienced a 266% increase in cervical stretch from collagen fiber stiffness value associated with the not remodeled cervix (Table 11).

TABLE 11
Uterine and cervical stretch patterns as cervical fiber stiffness is varied

| | Cervical collagen fiber stiffness ξ | | | | |
|---|---|---|---|---|---|
| | 1.71 kPa | 7.88 kPa | 36.3 kPa | 167 kPa | 769 kPa |
| Cervical stretch | 92.6% | 87.0% | 55.1% | 38.4% | 25.3% |

1st principal right stretch for the baseline geometry evaluated with a cervical fiber stiffness (ξ) value of 1.71, 7.89, 36.3, 167, and 769 kPa. The percentage reports the volume fraction of the cervical internal os above a 1.05 stretch. The membrane was removed for clarity.

### Discussion

This work develops a method to generate a patient-specific finite element model of the uterus, cervix, fetal membrane, and surrounding anatomy derived from maternal ultrasound scans. For a baseline investigation, a pregnant patient at 25 weeks of gestation was modeled and ultrasound measurements were converted into a parametric computer model. With this computer model, and basic engineering assumptions on previously published data, tissue stretch was assessed at various levels of intrauterine pressure (IUP). Results indicated that the distribution and magnitude of stretch of the cervix were affected by uterine wall mechanics, where direction and magnitude of cervical stretch were pulled towards uterine wall tension. The results show a stretch concentration at internal os of the cervix, matching findings from previous finite element models.

The flexibility of this model was also demonstrated by investigating the effects of cervical angle, length, offset, and material properties on the stretch generated at the internal os due to contraction-magnitude IUP. The sensitivity study of cervical structural parameters indicated that the effect of geometric parameters was magnified for a soft cervix and that cervical tissue stretching was most sensitive to posterior cervical offset (PCO) (Table 6). In this preliminary model, cervical stretch was reduced if the cervical canal was aligned with the uterine axis, where there the cervical axis was collinear with the uterine axis.

The level of tissue stretch at the IUP for 25 weeks of gestation was minimal. Tissue tensile stretch levels remained below 1.044 for the entire model at this gestational age. In comparing the results to previous studies (see, Fernandez, M., et al., 2015. “Investigating the mechanical function of the cervix during pregnancy using finite element models derived from high-resolution 3D MRI.”. Comput Methods Biomech Biomed Engin, May, pp. 1-14), larger tensile stretches were observed due to different fetal membranes adhesion scenarios. In the previous study, the fetal membranes were only tied to the lower uterine segment, whereas in this study, they are tied to both the lower uterine segment and upper cervix. Minimal tissue loading is expected at the 25 week gestation time-point considering the uterus grows and stretches to accommodate the enlarging amniotic sac. Uterine mass grows from 70 g to 1100 g and its volume capacity goes from 10 mL to 5 L. Early histologic, x-ray, and amniotic cavity pressure catheter studies offer the most complete view of pregnant uterine anatomy. From these data it was shown that in the first 12 weeks of pregnancy, hormonal signals initiate a considerable uterine growth process under negligible mechanical loading. From 12 to 16 weeks, the lower section of the uterine corpus unfolds into the lower uterine segment to allow for expansion of the amniotic sac without stretching the uterine wall. X-ray data of pregnant anatomy confirms the uterine wall thickness stays constant until 16 weeks and then begins to thin and elongate along its diameters as the fetus begins its rapid growth between 16 and 24 weeks. During this time, the uterus both grows and stretches. After 24 weeks, x-ray and ultrasonic evidence support that the uterus stops growing and continues to stretch and thin considerably until term.

Evidence from ex vivo cervical fibroblast studies suggest that cervical tissue stretch controls cervical material modeling processes. Hence, it is postulated that excessive cervical tissue stretch triggers premature cervical remodeling and possibly preterm birth. To evaluate the effect of cervical geometric parameters on cervical tissue stretch, model outcome variables were evaluated at a contraction-level IUP. Cervical tissue stretch was most sensitive to posterior cervical offset (PCO, Table 10) and is least sensitive to anterior uterocervical angle (AUCA, Table 8). Cervical tissue stretch was only sensitive to cervical length (CL, Table 9) if the cervix had already remodeled and was soft.

### Comparison to MRI-Based Model

To facilitate the creation of a more clinically applicable simulation and to reduce computational needs, the herein disclosed analytical method was used to create a pregnant anatomy. To understand how these simplifications effect cervical tissue stretch patterns, a parameterized model was compared with a model generated from previously published MRI-segmentation methods (as described in Fernandez, M., et al., 2015. “Investigating the mechanical function of the cervix during pregnancy using finite element models derived from high-resolution 3D MRI.” Comput Methods Biomech Biomed Engin, May, pp. 1-14). Briefly, dimensions of the uterus and cervix were measured from MRI data of the normal subject presented in Fernandez, M., et al., and these measurements were implemented in the herein parameterized procedure. Since this modeling method includes simplifications, the parameterized model does not contain bumps, divots, and variations in thickness that the segmented geometry includes. The tissue stretch patterns between the MRI-segmented and analytical geometries were compared. Overall, after application of intrauterine pressure of 0.817 kPa, the parameterized model predicts similar locations for strain concentration patterns as the MRI-segmented model (data not shown). The largest differences occurred at the site of a geometric feature in the MRI-based model at the location of the posterior internal os. At this location, the top of the cervix protrudes slightly into the volume of the uterus. Despite the differences between the results of each method, the parameterized model is an important tool in bridging the gap between future numerical clinical tools and the current clinical state of the art due to its unlimited flexibility and much reduced patient measurement to simulation timeline.

### Conclusion

Presented herein is a method for incorporating simplified anatomical geometries, fetal membrane contact conditions, IUP interaction, and cervical material properties into a mechanical simulation of pregnancy. In this study, the 1st principal right stretch was calculated under contraction-magnitude IUP levels in sonographically estimated FE models of pregnancy. Various cervical structural parameters were varied over a physiological range to analyze sensitivity of each dimension on cervical stretch. AUCA, CL, PCO, and cervical stiffness were analyzed, and the results show that a combination of AUCA, PCO, and cervical stiffness was the most significant LUS dimension measurement affecting the mechanical stretch state within the cervix, particularly near the internal os.

Numerous changes and modifications may be made to the aspects of the disclosure described herein and such changes and modifications can be made without departing from the spirit of the disclosure. It is, therefore, intended that the appended claims cover all such equivalent variations as fall within the true spirit and scope of the disclosure.

The disclosures of each patent, patent application, and publication cited or described in this document are hereby incorporated herein by reference, in its entirety.

### EMBODIMENTS

The following list of embodiments is intended to complement, rather than displace or supersede, the previous descriptions.

### Embodiment 1

A pessary configured to prevent a preterm birth in a patient, the pessary comprising a pessary body having:
a first end and a second end that are offset from one another, the first end defining a first opening;
an exterior wall that extends from the first end towards the second end, the exterior wall having an outer surface and an inner surface opposite the outer surface, the inner surface enclosing a channel that extends between the first and second ends and that is in fluid communication with the first opening; and
an interior wall that extends from the first end towards the second end into the channel, the interior wall having an inner surface configured to engage a cervix of the patient so as to secure the pessary to the cervix.

### Embodiment 2

The pessary of embodiment 1, wherein the interior wall has an outer surface that faces the exterior wall and an inner surface opposite the outer surface of the interior wall.

### Embodiment 3

The pessary of embodiment 2, wherein the pessary defines a space between the outer surface of the interior wall and the inner surface of the exterior wall.

### Embodiment 4

The pessary of any one of embodiments 1 to 3, wherein the interior wall encloses a second channel, the second channel in fluid communication with the channel.

### Embodiment 5

The pessary of embodiment 4, wherein the second channel tapers inward as it extends towards the second end.

### Embodiment 6

The pessary of any one of embodiments 1 to 5, wherein the interior wall includes a lower free end, and the interior wall extends from the first end and terminates at the lower free end.

### Embodiment 7

The pessary of any one of embodiments 1 to 5, wherein the pessary body defines an enclosed void between the interior wall and the external wall.

### Embodiment 8

The pessary of any one of embodiments 1 to 7, wherein the first end defines a first plane and the second end defines a second plane, the first and second planes being angularly offset from one another by an acute angle.

### Embodiment 9

The pessary of any one of embodiments 1 to 8, wherein the second end has a perimeter that has an oblong shape.

### Embodiment 10

The pessary of any one of embodiments 1 to 9, wherein the exterior wall flares out as it extends toward the second end.

### Embodiment 11

The pessary of any one of embodiments 1 to 10, wherein the first end has a first cross-sectional dimension, and the second end has a second cross-sectional dimension that is greater than the first cross-sectional dimension.

### Embodiment 12

The pessary of any one of embodiments 1 to 11, wherein the first opening is sized to receive the cervix.

### Embodiment 13

The pessary of any one of embodiments 1 to 12, wherein the inner surface of the interior wall defines at least one ridge that extends therefrom, the at least one ridge configured to engage the cervix.

### Embodiment 14

The pessary of any one of embodiments 1 to 13, wherein at least one recess extends into the inner surface of the interior wall, the at least one recess configured to receive a portion of the cervix as the cervix expands into the recess.

### Embodiment 15

The pessary of any one of embodiments 1 to 14, wherein the pessary body includes a first portion and a separate second portion, the separate second portion being configured to couple to the first portion.

### Embodiment 16

The pessary of embodiment 15, wherein the first portion includes the first end and the second portion includes the second end.

### Embodiment 17

The pessary of any one of embodiments 1 to 15, wherein the pessary defines a cervical correction angle between about −20° and about 20°.

### Embodiment 18

The pessary of any one of embodiments 1 to 16, wherein the first end of the pessary body defines a first plane, and the first plane is configured to form an angle with a central axis of the cervix in a range between 80° and 100° when the pessary is supported by the cervix.

### Embodiment 19

The pessary of any one of embodiments 1 to 18, further comprising a progesterone coating.

### Embodiment 20

The pessary of any one of the embodiments 1 to 19, wherein the pessary body comprises silicone or rubber.

### Embodiment 21

A pessary configured to prevent a preterm birth in a patient, the pessary comprising a pessary body having:
a first end that defines a first opening configured to receive a cervix of the patient;
a second end that is offset from the first end, the second end having a perimeter that has an oblong shape;
an interface surface that extends between the first end and the second end, the interface surface being configured to engage the cervix so as to secure the pessary to the cervix; and
an exterior wall that extends from the first end towards the second end, the exterior wall having an outer surface, and an inner surface opposite the outer surface, the inner surface enclosing a channel that extends between the first and second ends and that is in fluid communication with the first opening.

### Embodiment 22

The pessary of embodiment 21, wherein the first end defines a first plane and the second end defines a second plane, the first and second planes being angularly offset from one another by an acute angle.

### Embodiment 23

The pessary of any one of embodiments 21 and 22, wherein the exterior wall flares out as it extends towards the second end.

### Embodiment 24

The pessary of any one of embodiments 21 to 23, wherein the first end has a first cross-sectional dimension, and the second end has a second cross-sectional dimension that is greater than the first cross-sectional dimension.

### Embodiment 25

The pessary of any one of embodiments 21 to 24, wherein the interface surface defines at least one ridge that extends therefrom, the at least one ridge configured to engage the cervix.

### Embodiment 26

The pessary of any one of embodiments 21 to 25, wherein at least one recess extends into the interface surface, the at least one recess configured to receive a portion of the cervix as the cervix expands into the recess.

### Embodiment 27

A pessary configured to prevent a preterm birth in a patient, the pessary comprising a pessary body having:
a first end that defines a first opening configured to receive a cervix of the patient, the first end further defining a first plane;
a second end that is offset from the first end, the second end defining a second plane that is angularly offset from the first plane by an acute angle;
an interface surface extends between the first end and the second end, the interface surface configured to engage the cervix so as to secure the pessary to the cervix; and
an exterior wall that extends from the first end towards the second end, the exterior wall having an outer surface, and an inner surface opposite the outer surface, the inner surface enclosing a channel that extends between the first and second ends and that is in fluid communication with the first opening.

### Embodiment 28

The pessary of embodiment 27, wherein the exterior wall flares out as it extends towards the second end.

### Embodiment 29

The pessary of any one of embodiments 27 and 28, wherein the pessary body has a first height along a first side of the pessary body from the first end to the second end, and a second height along a second side of the pessary body from the first end to the second end, the first height being greater than the second height.

### Embodiment 30

The pessary of any one of embodiments 27 to 30, wherein:

the first opening defines a first central axis;

the second end defines a second opening; and

the second opening defines a second central axis that is angularly offset from the first central axis.

### Embodiment 31

A method of forming a pessary configured to prevent a preterm birth in a patient, the method comprising steps of:
obtaining a measurement of at least two of:
   (i) a first angle defined from a uterine longitudinal axis to a cervical axis, the uterine longitudinal axis being defined by a length of a uterus of the patient and the cervical axis being defined by a line that extends along a cervical opening of the patient;
   (ii) a cervical diameter of a cervix of the patient; and
   (iii) at least one height of a vaginal canal of the patient;
forming the pessary based on two or more of the first angle, the cervical diameter, and the at least one height, the pessary having a pessary body having:
   a first end and a second end offset from one another, the first end defining a first opening that is configured to receive the cervix;
an exterior wall that extends from the first end towards the second end, the exterior wall having an outer surface, and an inner surface opposite the outer surface, the inner surface enclosing a channel that extends between the first and second ends and that is in fluid communication with the first opening; and
an interface surface extends between the first end and the second end, the interface surface configured to engage the cervix so as to secure the pessary to the cervix.

### Embodiment 32

The method of embodiment 31, wherein the forming step comprises forming the pessary using additive manufacturing.

### Embodiment 33

The method of embodiment 31, wherein the forming step comprises:
selecting both i) a first portion of the pessary that includes the first end, and ii) a second portion of the pessary that includes the second end,
the selecting being based on two or more of the first angle, the cervical diameter, and the at least one height, and
the second portion being separate from the first portion.

### Embodiment 34

The method of embodiment 33, further comprising coupling the first and second portions to one another.

### Embodiment 35

The method of any one of embodiments 31 to 34, further comprising a step of inserting the pessary into the patient.

### Embodiment 36

The method of any one of embodiments 31 to 35, wherein the obtaining step comprises measuring the at least two of the first angle, the cervical diameter, and the at least one height.

### Embodiment 37

The method of any one of embodiments 31 to 35, wherein the obtaining step comprises receiving the at least two of the first angle, the cervical diameter, and the at least one height at a computing device.

### Embodiment 38

The method of embodiment 37, comprising generating a computer model of the pessary based on the at least two of the first angle, the cervical diameter, and the at least one height at a computing device.

### Embodiment 39

The method of embodiment 38, comprising forming the pessary based on the computer model.

### Embodiment 40

The method of embodiment 39, comprising forming the pessary using additive manufacturing based on the computer model.

### Embodiment 41

A method of predicting a likelihood of preterm birth in a patient, comprising:
generating a computer simulation of a mechanical environment of pregnancy from a series of maternal anatomical measurements, the maternal anatomical measurements being derived from a series of ultrasound-based images; and
predicting a likelihood of preterm birth if one or more factors characteristic of increased risk of preterm birth are present in the computer simulation.

### Embodiment 42

A method of preventing preterm birth in a subject comprising:
administering the pessary of any one of embodiments 1-30 to the subject, wherein one or more factors characteristic of increased risk of preterm birth are present in a computer simulation of a mechanical environment of pregnancy, and wherein the computer simulation of a mechanical environment of pregnancy is generated from a series of maternal anatomical measurements derived from a series of ultrasound-based images.

### Embodiment 43

The method of embodiment 41 or 42, wherein the factors characteristic of increased risk of preterm birth comprise a short cervix.

### Embodiment 44

The method of embodiment 43, wherein the short cervix is less than 25 mm in length.

### Embodiment 45

A method of predicting an amount of cervical stretch likely to occur in a subject during pregnancy, comprising:
performing ultrasound-based imaging of the subject to obtain a series of maternal anatomical measurements;
generating a computer simulation of a mechanical environment of pregnancy from the series of maternal anatomical measurements, the computer simulation representing a base-line for the patient;
applying a intrauterine pressure within the computer simulation of the mechanical environment; and
predicting the amount of cervical stretch that would result from the intrauterine pressure.

### Embodiment 46

A method of preventing cervical stretch in a subject during pregnancy comprising:
administering the pessary of any one of embodiments 1-30 to the subject, wherein, upon application of an interuterine pressure to a computer simulation of a mechanical environment of pregnancy, the computer simulation indicates that cervical stretch is likely to occur.

### Embodiment 47

The method of embodiment 46, wherein the computer simulation of a mechanical environment of pregnancy is generated from a series of maternal anatomical measurements derived from a series of ultrasound-based images.

### Embodiment 48

The method of any one of embodiments 41-45 or 47, wherein the maternal anatomical measurements comprise ultrasound-based images of one or more of placenta location, placenta volume, fetal biometrics, amniotic fluid index, uterine diameter, uterine thickness, cervical length, cervical diameter, cervical angle with anterior lower uterine segment, cervical os, mechanical load, and tissue stretch.

### Embodiment 49

A computer simulation of a mechanical environment of pregnancy comprising:
a memory adapted to store computer instructions;
a database; and
a processor adapted to process the computer instructions to implement a computer simulation of a mechanical environment of pregnancy, wherein the computer simulation of a mechanical environment of pregnancy comprises one or more measurements of a longitudinal uterine diameter, an anterior-posterior diameter, a cervical os offset from the longitudinal diameter, a transverse uterine diameter, a uterine wall thicknesses, a cervical length, a cervical diameter, a canal width, an angle with the anterior lower uterine segment, and an angle with periosteum of the symphysis pubis.

### Embodiment 50

The computer simulation of embodiment 49 for use in predicting a likelihood of preterm birth in a subject, the predicting comprising determining if one or more factors characteristic of increased risk of preterm birth are present in the computer simulation.

### Embodiment 51

The computer simulation of embodiment 50, wherein the factors characteristic of increased risk of preterm birth comprise a short cervix.

### Embodiment 52

The computer simulation of embodiment 51, wherein the short cervix is less than 25 mm in length.

### Embodiment 53

The computer simulation of embodiment 49 for use in predicting if cervical stretch is likely to occur in a subject during pregnancy, comprising applying an intrauterine pressure to the computer simulation and predicting the amount of cervical stretch that would result from the intrauterine pressure.

### Embodiment 54

The computer simulation of any one of embodiments 49-53, wherein the one or more measurements are derived from placenta location, placenta volume, fetal biometrics, amniotic fluid index, uterine diameter, uterine thickness, cervical length, cervical diameter, cervical angle with anterior lower uterine segment, cervical os, mechanical load, and tissue stretch.

### Embodiment 55

A use of a computer simulation of a mechanical environment of pregnancy for preventing preterm birth in a subject comprising:
predicting a likelihood of preterm birth in the subject if one or more factors characteristic of increased risk of preterm birth are present in the computer simulation, wherein the computer simulation is derived from a series of ultrasound-based images; and
if one or more factors characteristic of increased risk of preterm birth are present, administering any of the pessaries disclosed herein to the subject.

### Embodiment 56

The use of embodiment 55, wherein the factors characteristic of increased risk of preterm birth comprise a short cervix.

### Embodiment 57

The use of embodiment 56, wherein the short cervix is less than 25 mm in length.

### Embodiment 58

A use of a computer simulation of a mechanical environment of pregnancy for preventing cervical stretch in a subject during pregnancy comprising:
applying an intrauterine pressure to the computer simulation of the mechanical environment of pregnancy; and
predicting the amount of cervical stretch that would result from the intrauterine pressure, and, if cervical stretch is likely to occur, administering the pessary of any one of embodiments 1-30 to the subject.

### Embodiment 59

The use of embodiment 58, wherein the computer simulation of the mechanical environment of pregnancy is generated from a series of maternal anatomical measurements derived from ultrasound-based imaging of the subject.

### Embodiment 60

The use of any one of embodiments 55-57 or 59, wherein the ultrasound-based imaging comprises one or more of placenta location, placenta volume, fetal biometrics, amniotic fluid index, uterine diameter, uterine thickness, cervical length, cervical diameter, cervical angle with anterior lower uterine segment, cervical os, mechanical load, and tissue stretch.

What is claimed:

1. A pessary configured to prevent a preterm birth in a patient, the pessary comprising a pessary body having:
a first end and a second end that are offset from one another, the first end defining a first opening;
an exterior wall that extends from the first end towards the second end, the exterior wall having an outer surface and an inner surface opposite the outer surface,
the inner surface enclosing a channel that extends between the first and second ends and that is in fluid communication with the first opening; and
an interior wall that extends from the first end towards the second end into the channel, the interior wall having an inner surface configured to engage a cervix of the patient so as to secure the pessary to the cervix.

2. The pessary of claim 1, wherein the interior wall has an outer surface that faces the exterior wall and an inner surface opposite the outer surface of the interior wall.

3. The pessary of claim 2, wherein the pessary defines a space between the outer surface of the interior wall and the inner surface of the exterior wall.

4. The pessary of claim 1, wherein the interior wall encloses a second channel, the second channel in fluid communication with the channel.

5. The pessary of claim 4, wherein the second channel tapers inward as it extends towards the second end.

6. The pessary of claim 1, wherein the exterior wall flares out as it extends toward the second end.

7. The pessary of claim 1, wherein the first end has a first cross-sectional dimension, and the second end has a second cross-sectional dimension that is greater than the first cross-sectional dimension.

8. The pessary of claim 1, wherein the inner surface of the interior wall defines at least one ridge that extends therefrom, the at least one ridge configured to engage the cervix.

9. The pessary claim 1, wherein at least one recess extends into the inner surface of the interior wall, the at least one recess configured to receive a portion of the cervix as the cervix expands into the recess.

10. The pessary of claim 1, wherein the pessary defines a cervical correction angle between about −20° and about 20°.

11. The pessary of claim 10, wherein the first end of the pessary body defines a first plane, and the first plane is configured to form an angle with a central axis of the cervix in a range between about 80° and about 100° when the pessary is supported by the cervix.

12. A pessary configured to prevent a preterm birth in a patient, the pessary comprising a pessary body having:
a first end that defines a first opening configured to receive a cervix of the patient;
a second end that is offset from the first end, the second end having a perimeter that has an oblong shape;
an interface surface that extends between the first end and the second end, the interface surface being configured to engage the cervix so as to secure the pessary to the cervix; and
an exterior wall that extends from the first end towards the second end, the exterior wall having an outer surface, and an inner surface opposite the outer surface, the inner surface enclosing a channel that extends between the first and second ends and that is in fluid communication with the first opening.

13. The pessary of claim 12, wherein the exterior wall flares out as it extends towards the second end.

14. The pessary of claim 12, wherein the first end has a first cross-sectional dimension, and the second end has a second cross-sectional dimension that is greater than the first cross-sectional dimension.

15. The pessary of claim 12, wherein the interface surface defines at least one ridge that extends therefrom, the at least one ridge configured to engage the cervix.

16. The pessary of claim 12, wherein at least one recess extends into the interface surface, the at least one recess configured to receive a portion of the cervix as the cervix expands into the recess.

17. A pessary configured to prevent a preterm birth in a patient, the pessary comprising a pessary body having:
a first end that defines a first opening configured to receive a cervix of the patient, the first end further defining a first plane;
a second end that is offset from the first end, the second end defining a second plane that is angularly offset from the first plane by an acute angle;
an interface surface extends between the first end and the second end, the interface surface configured to engage the cervix so as to secure the pessary to the cervix; and
an exterior wall that extends from the first end towards the second end, the exterior wall having an outer surface, and an inner surface opposite the outer surface, the inner surface enclosing a channel that extends between the first and second ends and that is in fluid communication with the first opening.

18. The pessary of claim 17, wherein the exterior wall flares out as it extends towards the second end.

19. The pessary of claim 17, wherein the pessary body has a first height along a first side of the pessary body from the first end to the second end, and a second height along a second side of the pessary body from the first end to the second end, the first height being greater than the second height.

20. The pessary of claim 17, wherein:
the first opening defines a first central axis;
the second end defines a second opening, and the second opening defines a second central axis that is angularly offset from the first central axis.
